(19) United States
(12) Patent Application Publication
(10) Pub. No.: US 2025/0346908 A1
(43) Pub. Date: Nov. 13, 2025

(54) FLUOROGENIC NUCLEOSIDES

(71) Applicant: PRESIDENT AND FELLOWS OF HARVARD COLLEGE, Cambridge, MA (US)

(72) Inventors: Jonathan Rittichier, Cambridge, MA (US); Daniel J Wiegand, Cambridge, MA (US); Howon Lee, Cambridge, MA (US); Erkin Kuru, Cambridge, MA (US); George M. Church, Cambridge, MA (US)

(73) Assignee: PRESIDENT AND FELLOWS OF HARVARD COLLEGE, Cambridge, MA (US)

(21) Appl. No.: 18/869,037

(22) PCT Filed: May 25, 2023
(86) PCT No.: PCT/US2023/023484

§ 371 Date: Nov. 25, 2024

Related U.S. Application Data
(60) Provisional application No. 63/346,507, filed on May 27, 2022.

(51) Int. Cl. C12N 15/115; C07H 19/10; C07H 19/20; C12P 19/34; G01N 33/53; G01N 33/58

(52) U.S. Cl. CPC C12N 15/115; C07H 19/10; C07H 19/20; C12P 19/34; G01N 33/5308; G01N 33/582; C12N 2310/16

(57) ABSTRACT

Provided herein are Anorogenic nucleosides (e.g., Anorogenic nucleoside triphosphates (NTPs), e.g., Anorogenic reversible terminator nucleoside triphosphates) which can be used in the synthesis of Anorogenic oligonucleotides (e.g., Anorogenic DNA or RNA oligonucleotides, such as Anorogenic RNA aptamers). The Anorogenic oligonucleotides (e.g., Anorogenic DNA or RNA oligonucleotides, such as, Anorogenic RNA aptamers) can be used as Anorogenic probes to detect targets (e.g., antigens, biomarkers).

### RELATED APPLICATIONS

This application claims priority under 35 U.S.C. § 119(e) to U.S. Provisional Patent Application, U.S. Ser. No. 63/346,507, filed May 27, 2022, the entire contents of which is incorporated herein by reference.

### GOVERNMENT SUPPORT

This invention was made with government support under DE-FG02-02ER63445 awarded by U.S. Department of Energy (DOE). The government has certain rights in this invention.

### BACKGROUND

A common drawback to labeling molecular targets such as small molecules, proteins, and nucleic acids with fluorescent probes is the general inability to observe real-time labeling due to high background fluorescence. In many instances, washing steps are required for labeling protocols. DNA and RNA oligonucleotides are commonly labeled with fluorescent tags and are used in a wide array of applications, such as sequencing (e.g., next generation sequencing (NGS), fluorescent in-situ sequencing), biosensing/biomarker sensing (e.g., aptamers), drug delivery/localization (e.g., oligonucleotide therapeutics, mRNA therapeutics/vaccines), and microscopy/imaging (e.g., super-resolution microscopy, in situ imaging). However, due to drawbacks such as high background fluorescence, DNA and RNA oligonucleotides labeled with fluorescent tags are often poor imaging agents and often fail to provide the desired results.

### SUMMARY

Fluorogenic probes are a class of chemical sensors that undergo a change (e.g., increase) in their fluorescence emission intensity and/or fluorescence lifetime upon the occurrence of a particular physical or chemical event (i.e., they are “conditionally fluorescent”). Examples of such events are target binding or local solvent dipole or viscosity change. In one aspect, provided herein are fluorogenic nucleosides, including fluorogenic nucleoside triphosphates (NTPs) (e.g., fluorogenic reversible terminator nucleoside triphosphates), which can be used in the synthesis of fluorogenic oligonucleotides (e.g., fluorogenic DNA or RNA oligonucleotides, such as fluorogenic RNA aptamers). The fluorogenic oligonucleotides (e.g., fluorogenic DNA or RNA oligonucleotides, e.g., fluorogenic RNA aptamers) can be used as fluorogenic probes to detect targets (e.g., antigens, biomarkers).

The following are examples of fluorogenic reversible terminator NTPs provided herein:

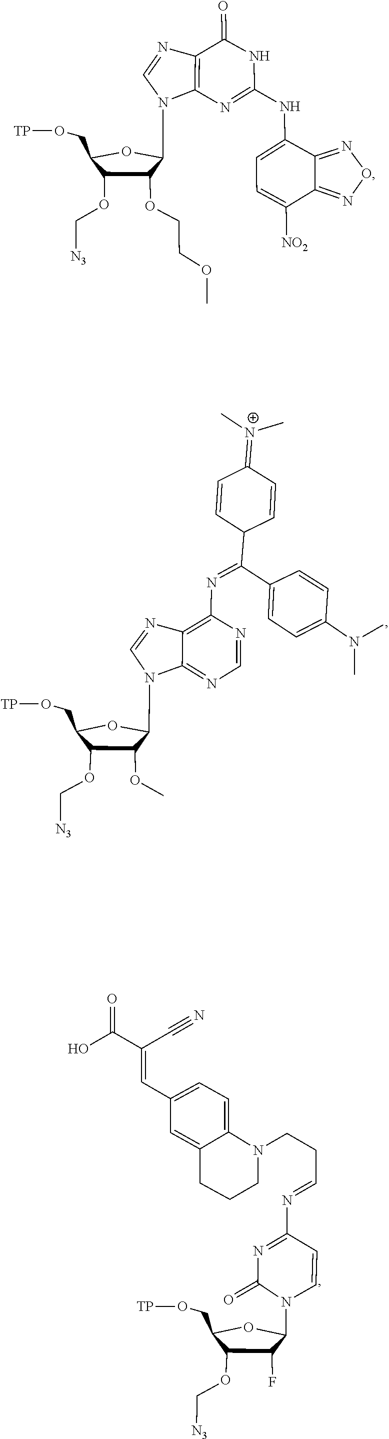

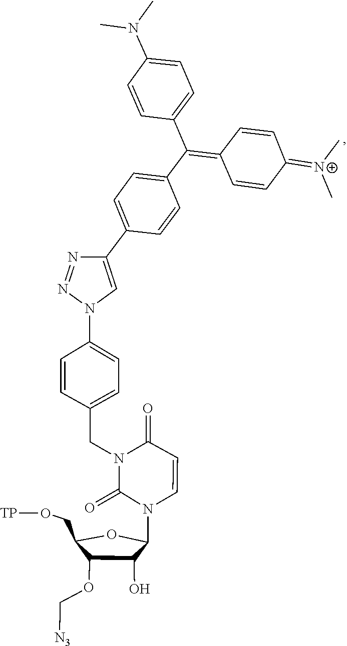

and salts and tautomers thereof, wherein TP is a triphosphate group.

### Definitions

### General Definitions

The following definitions are general terms used throughout the present application.

The term “fluorogenic” refers to a molecular entity (e.g., small molecule or oligonucleotide) that is conditionally fluorescent, i.e., that exhibits a change (e.g., increase) in its fluorescence emission intensity and/or fluorescence lifetime upon the occurrence of a particular physical or chemical event. Examples of such events are protein binding or local solvent dipole or viscosity change. A target-binding molecule (e.g., a fluorogenic RNA or DNA aptamer) comprising a fluorogenic small molecule can be used to detect binding of the target-binding molecule to the target (e.g., to detect the presence of said target). The target-binding molecule may specifically bind the target. Upon binding of the target-binding molecule to the target, the fluorescence of the fluorogenic small molecule may increase or decrease, thereby “sensing” the target. In addition or alternatively, the fluorescence lifetime of the fluorogenic small molecule may detectably change. In other words, a change (e.g., in fluorescence or change in fluorescence lifetime of the target-binding molecule (e.g., a fluorogenic RNA aptamer) is indicative of binding of the target-binding molecule to the target, and therefore indicative of the presence of the target.

The term “fluorogenic small molecule” refers to a small molecule that is fluorogenic, i.e., conditionally fluorescent.

“Fluorescence” is the visible or invisible emission of light by a substance that has absorbed light or other electromagnetic radiation. It can be measured, e.g., by fluorescence microscopy. In certain embodiments, fluorescence is visible and can be detected by the naked eye. In certain embodiments, the detection is colorimetric.

Fluorogenic NTPs and oligonucleotides provided herein have distinct fluorescence lifetime signatures, which can be detected, e.g., by a fluorescence lifetime microscopy. “Fluorescence lifetime” (FLT) is the time a fluorophore spends in the excited state before emitting a photon and returning to the ground state. Similar to fluorescence intensity, fluorogenic NTPs and oligonucleotides provided herein can also significantly change their fluorescence lifetimes based on the microenvironment they are in. For example, when a fluorogenic NTP or oligonucleotide is free in solution and unconstrained, it may be “darker” and typically will have a shorter fluorescence lifetime. On the other hand, when the fluorogenic NTP or oligonucleotide is physically restricted (e.g., in higher viscosity environments and/or upon binding to a target), it may become brighter and/or show a signature, longer fluorescence lifetime.

The term “target” or “target molecule” are used interchangeably, and as used herein refer any molecule or molecular structure (e.g., protein, nucleic acid, small molecule) which is capable of being bound by an oligonucleotide (e.g., an aptamer, e.g., an RNA aptamer).

The term “small molecule” refers to molecules, whether naturally occurring or artificially created (e.g., via chemical synthesis) that have a relatively low molecular weight.

Typically, a small molecule is an organic compound (e.g., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible.

As used herein, the term “conjugated” when used with respect to two or more molecules, means that the molecules are physically associated or connected with one another, either directly (i.e., via a covalent bond) or via one or more additional moieties that serves as a linking agent (i.e., “linker”), to form a structure that is sufficiently stable so that the moieties remain physically associated under the conditions in which the structure is used, e.g., physiological conditions.

As used herein, the term “polymerase” generally refers to an enzyme that is capable of synthesizing RNA or DNA oligonucleotides. In some embodiments, a polymerase is capable of synthesizing an oligonucleotide in a template-dependent manner. In other embodiments, a polymerase is capable of synthesizing an oligonucleotide in a template-independent manner. In some embodiments, a polymerase is an RNA polymerase. In some embodiments, a polymerase is a DNA polymerase. In some embodiments, a polymerase is a reverse transcriptase. A polymerase may be derived from any source, e.g., recombinant polymerase, bacterial polymerase. In some embodiments, a polymerase is a poly(N) polymerases. In some embodiments, a polymerase is a poly(U), poly(A), poly(C), or poly(G) polymerase. In some embodiments, a polymerase is capable of adding a nucleotide, e.g., a nucleotide, to the 3′-end of an oligonucleotide, e.g., an initiator oligonucleotide. In some embodiments, a polymerase selectively adds a single nucleotide species, e.g., nucleotide comprising an uracil base in the case of poly(U) polymerases, to the 3′ end of an oligonucleotide, e.g., an initiator oligonucleotide.

As used herein, the term “RNA oligonucleotide” generally refers to a polymer of nucleotides, ribonucleotides, or analogs thereof. An RNA oligonucleotide can have any sequence. As used herein, an RNA oligonucleotide may have any three-dimensional structure, and may perform any function, known or unknown to one of skill in the art. An RNA oligonucleotide may be naturally occurring or synthetic. In some embodiments, a RNA oligonucleotide may be a messenger RNA (mRNA), a transfer RNA, ribosomal RNA, a short interfering RNA (siRNA), a short-hairpin RNA (shRNA), a micro-RNA (miRNA), a ribozyme, a recombinant oligonucleotide, a branched oligonucleotide, an isolated or synthetic RNA oligonucleotide of any sequence, a probe, and/or a primer. In some embodiments, an RNA oligonucleotide comprises nucleotides comprising naturally occurring bases, e.g., adenine or uracil. In some embodiments, an RNA oligonucleotide comprises non-naturally occurring or modified nucleotides, e.g., nucleotides comprising sugar modifications, base modifications, e.g., purine or pyrimidine modifications. In some embodiments, a RNA oligonucleotide comprises a combination of naturally, non-naturally occurring, and modified nucleotides. In some embodiments, a nucleotide may comprise at least one modified backbone or linkage, e.g., a phosphorothioates backbone or linkage. In some embodiments, a RNA oligonucleotide is single-stranded. In other embodiments, a RNA oligonucleotide is double-stranded. In some embodiments, an RNA oligonucleotide is synthesized via template-independent synthesis. In some embodiments, an RNA oligonucleotide is at least 5, at least 10, at least 20, at least 50, at least 100, at least 200, at least 300, at least 400, or at least 500 nucleotides in length. In some embodiments, an RNA oligonucleotide is from 5-500, 10-500, 20-500, 50-500, 100-500, 200-500, 300-500, or 400-500 nucleotides in length.

As used herein, the term “DNA oligonucleotide” generally refers to a polymer of DNA nucleotides, deoxyribonucleotides, or analogs thereof. As used herein, a DNA oligonucleotide may have any three-dimensional structure, and may perform any function, known or unknown to one of skill in the art. A DNA oligonucleotide may be naturally occurring or synthetic. In some embodiments, a DNA oligonucleotide may be an exon, an intron, a cDNA sequence, a recombinant oligonucleotide, a branched oligonucleotide, a plasmid, a vectors, and/or an isolated DNA of any sequence. In some embodiments, a DNA oligonucleotide comprise DNA nucleotides comprising naturally occurring bases, e.g., adenine, cytosine, guanine, or thymine. In some embodiments, a DNA oligonucleotide comprise non-naturally occurring or modified DNA nucleotides, e.g., DNA nucleotides comprising sugar modifications, purine or pyrimidine modifications. In some embodiments, a DNA oligonucleotide comprises a combination of naturally, non-naturally occurring, and modified DNA nucleotides. In some embodiments, a DNA nucleotide may comprise at least one modified backbone or linkage, e.g., a phosphorothioates backbone or linkage. In some embodiments, a DNA oligonucleotide is single-stranded. In other embodiments, a DNA oligonucleotide is double-stranded. In some embodiments, a DNA oligonucleotide is synthesized via reverse transcription. In some embodiments, a DNA oligonucleotide is at least 5, at least 10, at least 20, at least 50, at least 100, at least 200 DNA, at least 300, at least 400, or at least 500 DNA nucleotides in length. In some embodiments, an DNA oligonucleotide is from 5-500, 10-500, 20-500, 50-500, 100-500, 200-500, 300-500, or 400-500 nucleotides in length.

As used herein, the term “nucleoside” generally refers to a nucleotide monomer that comprises a ribose sugar linked to a nucleobase. A “nucleoside monophosphate” generally refers to a nucleotide monomer that comprises a ribose sugar linked to a nucleobase and phosphate group. A “nucleoside diphosphate” generally refers to a nucleotide monomer that comprises a ribose sugar linked to a nucleobase and a diphosphate group. A “nucleoside triphosphate” (“NTP”) generally refers to a nucleotide monomer that comprises a ribose sugar linked to a nucleobase and a triphosphate group.

As used herein, the term “initiator oligonucleotide” generally refers to a short, single-stranded RNA oligonucleotide that is capable of initiating template-independent synthesis. An initiator oligonucleotide is, in certain embodiments, less than 20 nucleotides in length. In some embodiments, an initiator oligonucleotide is less than 20, less than 18, less than 15, less than 12, less than 10, less than 8, or less than 5 nucleotides in length. In some embodiments, an initiator oligonucleotide is 5, 6, 7, 8, 9, 10, 11, 12, 13, 14, 15, 16, 17, 18, 19, or 20 nucleotides in length. In some embodiments, an initiator oligonucleotide is labeled at its 5′ end, e.g., labeled with a fluorophore. In some embodiments, an initiator oligonucleotide is attached to a substrate at its 5′-end. In some embodiments, a substrate may be a glass surface, a bead, a biomolecule, or any conceivable substrate suitable for template-independent synthesis.

As used herein, the term “template-independent” generally refers to the synthesis of an RNA oligonucleotide that does not require a template DNA oligonucleotide. Template-independent synthesis will generally comprise the use of an initiator oligonucleotide and a polymerase, e.g., a poly(N) polymerase. Oligonucleotides, e.g., RNA oligonucleotides, synthesized using template-independent synthesis are generally synthesized by adding nucleotides, e.g., nucleotides, to the 3′ end of an existing oligonucleotide, e.g., an initiator oligonucleotide.

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The disclosure additionally encompasses peptides as individual isomers substantially free of other isomers, and alternatively, as mixtures of various isomers.

The term “tautomers” or “tautomeric” refers to two or more interconvertible compounds resulting from at least one migration of a hydrogen atom or electron lone pair, and at least one change in valency (e.g., a single bond to a double bond or vice versa). The exact ratio of the tautomers depends on several factors, including temperature, solvent, and pH. Exemplary tautomerizations include keto-to-enol, amide-to-imide, lactam-to-lactim, enamine-to-imine, and enamine-to-(a different enamine) tautomerizations. Compounds described herein are provided in any and all tautomeric forms. Example of tautomers resulting from the delocalization of electrons (e.g., resonance structures) are shown below:

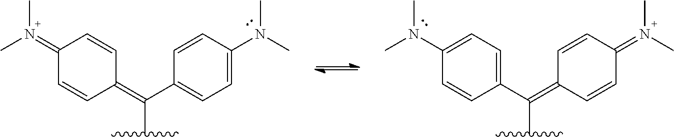

In a formula, the bond [symbol] is a single bond, the dashed line [symbol] is a single bond or absent, and the bond [symbol] or [symbol] is a single or double bond. Additionally, the bond [symbol] or [symbol] is a double or triple bond.

Unless otherwise provided, formulae and structures depicted herein include peptides that do not include isotopically enriched atoms, and also include peptides that include isotopically enriched atoms (“isotopically labeled derivatives”). For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such peptides are useful, for example, as analytical tools or probes in biological assays. The term “isotopes” refers to variants of a particular chemical element such that, while all isotopes of a given element share the same number of protons in each atom of the element, those isotopes differ in the number of neutrons.

When a range of values (“range”) is listed, it encompasses each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

The term “alkyl” refers to a radical of a straight-chain or branched saturated hydrocarbon group having from 1 to 20 carbon atoms (“C₁₋₂₀ alkyl”). In some embodiments, an alkyl group has 1 to 6 carbon atoms (“C₁₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like.

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, all of the haloalkyl hydrogen atoms are independently replaced with fluoro to provide a “perfluoroalkyl” group. In some embodiments, all of the haloalkyl hydrogen atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”).

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). The one or more carbon-carbon double bonds can be internal (such as in 2-butenyl) or terminal (such as in 1-butenyl). In an alkenyl group, a C═C double bond for which the stereochemistry is not specified (e.g., —CH═CHCH₃ or

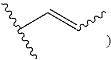

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 1 to 20 carbon atoms, at least one double bond, and 1 or more heteroatoms within the parent chain (“heteroC₁₋₂₀ alkenyl”).

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). The one or more carbon-carbon triple bonds can be internal (such as in 2-butynyl) or terminal (such as in 1-butynyl).

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 1 to 20 carbon atoms, at least one triple bond, and 1 or more heteroatoms within the parent chain (“heteroC₁₋₂₀ alkynyl”).

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 6 ring carbon atoms (“C₃₋₆ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur. In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom or the ring that does not contain a heteroatom.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A chemical moiety is optionally substituted unless expressly provided otherwise. Any chemical formula provided herein may also be optionally substituted. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, heteroaryl, acyl groups are optionally substituted. In general, the term “substituted” when referring to a chemical group means that at least one hydrogen present on the group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The disclosure is not limited in any manner by the exemplary substituents described herein.

Exemplary substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SCN, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl; wherein X⁻ is a counterion;
or two geminal hydrogens on a carbon atom are replaced with the group ═O, ═S, ═NN(R^{bb})₂, ═NNR^{bb}C(═O)R^{aa}—, ═NNR^{bb}C(═O)OR^{aa}, ═NNR^{bb}S(═O)₂R^{aa}, ═NR^{bb}, or ═NOR^{cc}; wherein:
   each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring;
   each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring;
   each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring; and each X⁻ is a counterion.

In certain embodiments, each substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —N₃, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

The term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S—SR^{cc}, —SC(═S)SR^{aa}, —SC(═S)OR^{aa}, —SC(═S) N(R^{bb})₂, —SC(═O)SR^{aa}, —SC(═O)OR^{aa}, —SC(═O)N(R^{bb})₂, and —SC(═O)R^{aa}, wherein R^{aa}, R^{bb}, and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group. The term “monosubstituted amino” refers to an amino group wherein the nitrogen atom directly attached to the parent molecule is substituted with one hydrogen and one group other than hydrogen, and includes groups selected from —NH(R^{bb}) —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen. The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen. The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “acyl” refers to a group having the general formula —C(═O)R^{aa}, —C(═O)OR^{aa}, —C(═O)—O—C(═O)R^{aa}, —C(═O)SR^{aa}, —C(═O)N(R^{bb})₂, —C(═S)R^{aa}, —C(═S)N(R^{bb})₂, and —C(═S)S(R^{aa}), —C(═NR^{bb})R^{aa}, —C(═NR^{bb})OR^{aa}, —C(═NR^{bb})SR^{aa}, and —C(═NR^{bb})N(R^{bb})₂, wherein R^{aa} and R^{bb} are as defined herein. Exemplary acyl groups include aldehydes (—CHO), carboxylic acids (—CO₂H), ketones, acyl halides, esters, amides, imines, carbonates, carbamates, and ureas.

A “counterion” or “anionic counterion” is a negatively charged group associated with a positively charged group in order to maintain electronic neutrality. An anionic counterion may be monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

As used herein, the term “salt” refers to any and all salts, and encompasses pharmaceutically acceptable salts. The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

These and other exemplary substituents are described in more detail in the Detailed Description, Examples, Figures, and Claims. The disclosure is not limited in any manner by the above exemplary listing of substituents.

### BRIEF DESCRIPTION OF THE DRAWINGS

The accompanying drawings, which constitute a part of this specification, illustrate several embodiments of the invention and together with the description, serve to explain the principles of the invention.

FIG. 1 shows an exemplary generic structure of fluorogenic reversible terminator nucleoside triphosphates (NTPs).

FIGS. 2A-2C show the use of template-independent, enzymatic oligonucleotide synthesis to prepare a “light up” oligonucleotide that is modified with a fluorogenic small molecule. FIG. 2A shows an overview of enzymatic oligonucleotide synthesis (EOS). One cycle consists of an extension (the addition of a reversible terminator NTP) followed by a mild deblocking step. FIG. 2B shows examples of building blocks used in EOS to prepare “light-up” aptamers. Upon the introduction of the target, the oligonucleotide rigidifies resulting in an enhancment of fluorescence (FIG. 2C).

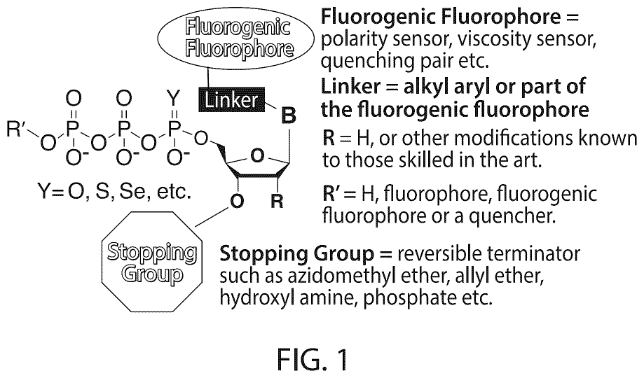

### DETAILED DESCRIPTION OF CERTAIN EMBODIMENTS

Provided herein are fluorogenic nucleosides, including fluorogenic nucleoside triphosphates (NTPs) (e.g., fluorogenic reversible terminator NTPs), which can be used in the synthesis of fluorogenically-labeled DNA and RNA oligonucleotides (e.g., fluorogenic RNA aptamers). The fluorogenic NTPs and fluorogenic oligonucleotides comprising them provided herein can change (e.g., increase) fluorescence emission intensity and/or fluorescence lifetime upon the occurrence of a particular physical or chemical event (i.e., they are “conditionally fluorescent”). Examples of such events are target binding or local solvent dipole or viscosity change. Therefore, the fluorogenic NTPs and fluorogenic oligonucleotides provided herein can be used in a variety of applications, such as sequencing (e.g., next generation sequencing (NGS), fluorescent in-situ sequencing), biosensing/biomarker sensing (e.g., aptamers), drug delivery/localization (e.g., oligonucleotide therapeutics, mRNA therapeutics/vaccines), and microscopy/imaging (e.g., super-resolution microscopy, in situ imaging).

### Fluorogenic Nucleoside Triphosphates (NTPs)

Provided herein are fluorogenic nucleosides comprising a fluorogenic small molecule. In certain embodiments, the fluorogenic nucleoside comprises a nucleoside monophosphate. In certain embodiments, the fluorogenic nucleoside comprises a nucleoside diphosphate. In certain embodiments, the fluorogenic nucleoside comprises a nucleoside triphosphate (NTP).

Provided herein are nucleoside triphosphates (NTPs) comprising a fluorogenic small molecule (i.e., “fluorogenic NTPs”). In certain embodiments, the NTP comprises a ribose nucleoside (i.e., the sugar moiety of the NTP is ribose or a modified ribose, i.e., a “ribonucleotide triphosphate”). As described herein, the fluorogenic NTPs provided are conditionally fluorescent.

The fluorogenic small molecule may be conjugated to the base, sugar, phosphate (e.g., mono-, di-, or triphosphate), or other moiety of the nucleoside. For example, the fluorogenic small molecule may be conjugated to the base, sugar, triphosphate, or other moiety of the NTP. In certain embodiments, the fluorogenic small molecule is conjugated to the base moiety of the NTP. In certain embodiments, the fluorogenic small molecule is conjugated to the NTP via a bond. In certain embodiments, the fluorogenic small molecule is conjugated to the NTP via a linker (e.g., a non-cleavable linker). In certain embodiments, the fluorogenic small molecule is conjugated to the base moiety of the NTP via a linker (e.g., a non-cleavable linker).

A fluorogenic nucleoside (e.g., fluorogenic NTP) provided herein may comprise any combination of the chemical groups or modifications described herein, including, but not limited to, the following. Other examples of nucleoside modifications are described in, e.g., International PCT Application Publication No. WO 2020/077227, published Apr. 16, 2020, the entire contents of which is incorporated herein by reference.

### Bases of Fluorogenic NTPs

The base moiety (i.e., nucleobase moiety) of a fluorogenic nucleoside (e.g., fluorogenic NTP) described herein can be a naturally occurring nucleobase (e.g., guanine (G), uracil (U), adenine (A), cytosine (C), thymine (T)). In other embodiments, the base moiety is a non-natural or modified nucleobase.

Non-limiting examples of modified nucleobases include, but are not limited to, 5-methylcytosine, pyridin-4-one, pyridin-2-one, phenyl, pseudouracil, 3-methyl uracil, dihydrouridine, naphthyl, aminophenyl, 5-alkylcytidines, 5-alkyluridines, 5-halouridines, 6-azapyrimidines, 6-alkylpyrimidines, propyne, quesosine, 2-thiouridine, 4-thiouridine, 4-acetyltidine, 5-(carboxyhydroxymethyl)uridine, 5-carboxymethylaminomethyl-2-thiouridine, 5-carboxymethylaminomethyluridine, β-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N₆-isopentenyladenosine, 3-D-mannosylqueosine, uridine-5-oxyacetic acid, 2-thiocytidine, and thymine derivatives.

Other non-limiting examples of nucleobases include, but are not limited to, natural or non-natural pyrimidines and purines; and may include, but are not limited to, N¹-methyl-adenine, N⁶-methyl-adenine, 8′-azido-adenine, N,N-dimethyl-adenosine, aminoallyl-adenosine, 5′-methyl-urdine, pseudouridine, N¹-methyl-pseudouridine, 5′-hydroxy-methyl-uridine, 2′-thio-uridine, 4′-thio-uridine, hypoxanthine, xanthine, 5′-methyl-cytidine, 5′-hydroxy-methyl-cytidine, 6′-thio-guanine, and N⁷-methyl-guanine.

In certain embodiments, the fluorogenic NTP comprises an NTP selected from the group consisting of N¹-methyladenosine-5′-triphosphate, N⁶-methyladenosine-5′-triphosphate, N⁶-methyl-2-aminoadenosine-5′-triphosphate, 5-methyluridine-5′-triphosphate, N¹-methylpseudouridine-5′-triphosphate, pseudouridine-5′-triphosphate, 5-hydroxymethyluridine-5′-triphosphate, 5-methylcytidine-5′-triphosphate, 5-hydroxymethylcytidine-5′-triphosphate, N⁷-methylguanosine-triphosphate, 8′-adizoadenisone-5′-triphosphate, inosine 5′-triphosphate, 2-thiouridine-5′-triphosphate, 6-thioguanosine-5′-triphosphate, 4-thiouridine-5′-triphosphate, and xanthosine-5′-triphosphate (i.e., any of the foregoing may be conjugated to a fluorogenic small molecule described herein). In certain embodiments, the fluorogenic NTP is derived from one of the foregoing NTPs.

### Sugars of Fluorogenic NTPs

A fluorogenic nucleoside (e.g., fluorogenic NTP) described herein may comprise a natural ribose or deoxyribose sugar moiety. In other embodiments, a fluorogenic NTP described herein comprises a non-natural or modified ribose or deoxyribose sugar moiety. In certain embodiments, the ribose or deoxyribose is modified (e.g., substituted) at the 1′, 2′, 3′, 4′, and/or 5′ position. In some embodiments, the fluorogenic NTP comprises a substituent at the 2′ position. In some embodiments, the fluorogenic NTP comprises a substituent at the 3′ position.

In some embodiments, the 2′ position of the sugar is substituted with a halogen, e.g., a fluorine group; an alkyl group, e.g., methyl or ethyl group; a methoxy group; an amino group; a thio group; an aminopropyl group; a dimethylaminoethyl; a dimethylaminopropyl group; a dimethylaminoethyloxyethyl group; an azido group; a silyl group; a cyclic alkyl group; or a N-methylacetamido group. In certain embodiments, the 2′ position of the is substituted with a hydroxyl (—OH), hydrogen (—H), fluoro (—F), amine (—NH₃), azido (—N₃), thiol (—SH), methoxy (—OCH₃), or methoxyethanol (—OCH₂CH₂OCH₃).

For example, in certain embodiments, the fluorogenic nucleoside (e.g., fluorogenic NTP) comprises a 2′-F, 2′-O-alkyl, 2′-amino, or 2′-azido nucleoside. In certain embodiments, the fluorogenic nucleoside (e.g., fluorogenic NTP) comprises a 2′-F nucleoside. In certain embodiments, the fluorogenic NTP comprises an NTP selected from the group consisting of 2′-fluoro-2′-deoxyadenosine-5′-triphosphate, 2′-fluoro-2′-deoxycytidine-5′-triphosphate, 2′-fluoro-2′-deoxyguanosine-5′-triphosphate, and 2′-fluoro-2′-deoxyuridine-5′-triphosphate (i.e., any of the foregoing may be conjugated to a fluorogenic small molecule described herein).

In certain embodiments, the fluorogenic nucleoside (e.g., fluorogenic NTP) comprises a 2′-O-alkyl nucleoside. In certain embodiments, the fluorogenic NTP comprises an NTP selected from 2′-O-methyladenosine-5′-triphosphate, 2′-O-methylcytidine-5′-triphosphate, 2′-O-methylguanosine-5′-triphosphate, 2′-O-methyluridine-5′-triphosphate, and 2′-O-methylinosine-5′-triphosphate (i.e., any of the foregoing may be conjugated to a fluorogenic small molecule described herein).

In certain embodiments, the fluorogenic nucleoside (e.g., fluorogenic NTP) comprises a 2′-O-amino substituted nucleoside. In certain embodiments, the fluorogenic NTP comprises an NTP selected from the group consisting of 2′-amino-2′-deoxycytidine-5′-triphosphate, 2′-amino-2′-deoxyuridine-5′-triphosphate, 2′-amino-2′-deoxyadenosine-5′-triphosphate, and 2′-amino-2′-deoxyguanosine-5′-triphosphate (i.e., any of the foregoing may be conjugated to a fluorogenic small molecule described herein).

In certain embodiments, the fluorogenic nucleoside (e.g., fluorogenic NTP) comprises a 2′-O-azido substituted nucleoside. In certain embodiments, the fluorogenic NTP comprises an NTP selected from the group consisting of 2′-azido-2′-deoxycytidine-5′-triphosphate, 2′-azido-2′-deoxyuridine-5′-triphosphate, 2′-azido-2′-deoxyadenosine-5′-triphosphate, and 2′-azido-2′-deoxyguanosine-5′-triphosphate (i.e., any of the foregoing may be conjugated to a fluorogenic small molecule described herein).

In some embodiments, the 3′ position of the sugar may be modified with a halogen, e.g., a fluorine group; an alkyl group, e.g., methyl or ethyl group; a methoxy group; an amino group; a thio group; an aminopropyl group; a dimethylaminoethyl; a dimethylaminopropyl group; a dimethylaminoethyloxyethyl group; an azido group; a silyl group; a cyclic alkyl group; or a N-methylacetamido group. In certain embodiments, the 3′ position of the sugar is modified with a hydroxyl (—OH), hydrogen (—H), fluoro (—F), amine (—NH₃), azido (—N₃), thiol (—SH), methoxy (—OCH₃), or methoxyethanol (—OCH₂CH₂OCH₃).

In certain embodiments the fluorogenic nucleoside (e.g., fluorogenic NTP) comprises an irreversible terminator group, also known as a capping nucleoside. In certain embodiments the fluorogenic NTP comprises a nucleoside selected from 3′-O-methyl-NTP, 3′-O-methyl-dNTP, 3′-azido-dNTP, 3′-azido-NTP, 3′-amine-dNTP, and 3′-amine-NTP (i.e., any of the foregoing may be conjugated to a fluorogenic small molecule described herein).

In certain embodiments, the fluorogenic nucleoside (e.g., fluorogenic NTP) is a 2′-modified reversible terminator RNA nucleotide (e.g., 2′-O-protected reversible terminator nucleotide). 2′-modified reversible terminator nucleotides are described herein. In certain embodiments, the fluorogenic nucleoside (e.g., fluorogenic NTP) is a 3′-modified reversible terminator RNA nucleotide (e.g., 3′-O-protected reversible terminator nucleotide). 3′-modified reversible terminator nucleotides are described herein.

Other modifications to the sugar are contemplated. These modifications include, but not limited to, replacing the ring's oxygen with a sulfur. In certain embodiments, a bridge is introduced between the 2′-carbon and the 4′-carbon (e.g., to limit ring conformation). In some embodiments, a modified nucleotide is a bridged nucleotide, e.g., locked nucleic acid (LNA); a constrained ethyl nucleotide (cEt), or an ethylene bridged nucleic acid (ENA) nucleotide.

In certain embodiments, the fluorogenic NTP is of the formula:

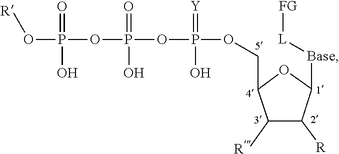

or a salt thereof, wherein:
Y is O, S, or Se;
R and R″′ are each independently is hydrogen, halogen, —CN, —NO₂, —N₃, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted hydroxyl, optionally substituted amino, or optionally substituted thiol;
Base is a natural or non-natural nucleotide base;
L is a bond or a linker;
FG is a fluorogenic small molecule; and
R′ is hydrogen, or a group comprising a fluorophore, fluorogenic small molecule, or fluorescent quencher.

In certain embodiments, R and/or R″′ are independently —OR^{P}, wherein each instance of R^{P} is independently an oxygen protecting group, optionally substituted acyl, or an amino acid. In certain embodiments, R and/or R″′ comprise a reactive moiety for bioconjugation (e.g., click chemistry handle, e.g., azide or alkyne), a fluorophore, catalytic protein, oligonucleotide, or reporting tag.

### Phosphate Moieties of Fluorogenic NTPs

As described herein, in certain embodiments, a fluorogenic nucleoside comprises a mono-, di-, or triphosphate group. In some embodiments, the fluorogenic nucleoside comprises a modified mono-, di-, or triphosphate group. In some embodiments, a fluorogenic NTP may comprise a modified triphosphate group, e.g., a phosphorothioate. Non-limiting examples of modified phosphate groups include phosphorothioates, phosphotriesters, methyl phosphonates, alkyl, heterocyclic, amide, morpholino, peptide nucleic acids (PNA), and other known phosphorus-containing groups. In certain embodiments, the modification is to the alpha (α) phosphate of the triphosphate. In certain embodiments, the nucleotide is an (α) thiophosphonate. In certain embodiments, the modifications to the beta (β) and/or gamma (γ) phosphates of the triphosphate.

### Fluorogenic Reversible Terminator NTPs

In certain embodiments, a fluorogenic NTP provided herein comprises a 2′- and/or 3′-reversible terminator group (i.e., “fluorogenic reversible terminator NTP”). A “reversible terminator” is a non-natural chemical moiety at the 2′- and/or 3′-position that is capable of being removed. In certain embodiments, the fluorogenic reversible terminator NTP is protected at the 2′-O— and/or 3′-O-positions with an oxygen protecting group.

In certain embodiments, the fluorogenic reversible terminator NTP is protected at the 2′-O-position with an oxygen protecting group (“2′-O-protected fluorogenic reversible terminator NTP”). In certain embodiments, the fluorogenic reversible terminator NTP is protected at the 3′-O-position with an oxygen protecting group (“3′-O-protected fluorogenic reversible terminator NTP”).

In certain embodiments, a 2′-O-protected fluorogenic reversible terminator NTP comprises a 2′-O-alkyl, 2′-O-silyl, 2′-O-allyl, 2′-O-azidomethyl, 2′-O-benzyl, 2′-O-coumarinyl, or a 2′-O-carbonate group. In certain embodiments, the 2′-O-protected fluorogenic reversible terminator NTP comprises a 2′-O-carbonate group selected from 2′-O-allyloxycarbonyl and 2′-O-(2-oxo-2H-chromen-4-yl)methyloxycarbonyl. In certain embodiments, a 2′-O-protected fluorogenic reversible terminator NTP comprises a 2′-O-allyl, 2′-O-azidomethyl, 2′-O-allyl carbonate, 2′-O-azidomethyl carbonate, or 2′-azidoethoxy group.

In certain embodiments, the 2′-O-protected fluorogenic reversible terminator NTP comprises 2′-O-allyl-NTP or 2′-O-azidomethyl-NTP (i.e., any of the foregoing may be conjugated to a fluorogenic small molecule described herein).

In certain embodiments, a 3′-O-protected fluorogenic reversible terminator NTP comprises a 3′-O-alkyl, 3′-O-silyl, 3′-O-allyl, 3′-O-azidomethyl, 3′-O-benzyl, 3′-O-coumarinyl, or a 3′-O-carbonate group. In certain embodiments, the 3′-O-protected fluorogenic reversible terminator NTP comprises a 3′-O-carbonate group selected from 3′-O-allyloxycarbonyl and 3′-O-(2-oxo-2H-chromen-4-yl)methyloxycarbonyl. In certain embodiments, a 3′-O-protected fluorogenic reversible terminator NTP comprises a 3′-O-allyl, 3′-O-azidomethyl, 3′-O-allyl carbonate, 3′-O-azidomethyl carbonate, or 3′-azidoethoxy group.

In certain embodiments, the 3′-O-protected fluorogenic reversible terminator NTP comprises 3′-O-allyl-NTP, 3′-O-azidomethyl-NTP, 3′-O-allyl carbonate-NTP, 3′-O-allyl carbonate-dNTP, 3′-O-azidomethyl carbonate-NTP, or 3′-O-azidomethyl carbonate-dNTP (i.e., any of the foregoing may be conjugated to a fluorogenic small molecule described herein).

In certain embodiments, the 3′-O-protected fluorogenic reversible terminator NTP comprises 3′-O-allyl-NTP, 3′-(O-allyl-carbonate)-dNTP (e.g., 3′-(O-allyl-carbonate)-dATP, etc.), 3′-(O-azidomethyl carbonate)-dNTP, 3′-(O-acetate)-dNTP, 3′-(O-acyl amino acids)-dNTP, 3′-(O-3-methylcoumarin)-dNTP, 3′-(O-(4-methylcoumarin carbonate)-dNTP, 3′-(O-(2-nitrobenzyl)-dNTP, 3′-(O-(2-nitrobenzyl carbonate)-dNTP, 3′-(O-TMS)-dNTP, or 3′-(O-Teoc)-dNTP (i.e., any of the foregoing may be conjugated to a fluorogenic small molecule described herein).

In certain embodiments, 3′-O-protected fluorogenic reversible terminator NTP comprises a 3′-O-amino acid group (e.g., comprising any standard or non-standard amino acid). In certain embodiments, the amino acid can be removed using an esterase.

As described herein, reversible terminator oligonucleotides may be protected with oxygen protecting groups (e.g., at the 2′-O and/or 3′-O position, e.g, R^{P} groups). Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference. Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). In certain embodiments, an oxygen protecting group is silyl. In certain embodiments, an oxygen protecting group is t-butyldiphenylsilyl (TBDPS), t-butyldimethylsilyl (TBDMS), triisoproylsilyl (TIPS), triphenylsilyl (TPS), triethylsilyl (TES), trimethylsilyl (TMS), triisopropylsiloxymethyl (TOM), acetyl (Ac), benzoyl (Bz), allyl carbonate, 2,2,2-trichloroethyl carbonate (Troc), 2-trimethylsilylethyl carbonate, methoxymethyl (MOM), 1-ethoxyethyl (EE), 2-methyoxy-2-propyl (MOP), 2,2,2-trichloroethoxyethyl, 2-methoxyethoxymethyl (MEM), 2-trimethylsilylethoxymethyl (SEM), methylthiomethyl (MTM), tetrahydropyranyl (THP), tetrahydrofuranyl (THF), p-methoxyphenyl (PMP), triphenylmethyl (Tr), methoxytrityl (MMT), dimethoxytrityl (DMT), allyl, p-methoxybenzyl (PMB), t-butyl, benzyl (Bn), allyl, or pivaloyl (Piv).

In certain embodiments, the fluorogenic reversible terminator NTP is of the formula:

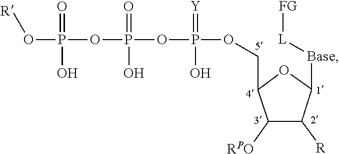

or a salt thereof, wherein:
Y is O, S, or Se;
R^{P} is an oxygen protecting group, optionally substituted acyl, or an amino acid;
R is hydrogen, halogen, —CN, —NO₂, —N₃, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted hydroxyl, optionally substituted amino, or optionally substituted thiol;
Base is a natural or non-natural nucleotide base;
L is a bond or a linker;
FG is a fluorogenic small molecule; and
R′ is hydrogen, or a group comprising a fluorophore, fluorogenic small molecule, or fluorescent quencher.

In certain embodiments, the fluorogenic reversible terminator NTP is of the formula:

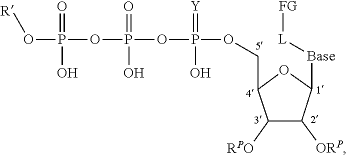

or a salt thereof, wherein:
Y is O, S, or Se;
each instance of R^{P} is hydrogen, an oxygen protecting group, optionally substituted acyl, or an amino acid, or two R^{P} are joined together with the intervening atoms to form optionally substituted heterocyclyl; provided that at least one R^{P} is an oxygen protecting group, optionally substituted acyl, or an amino acid; and
Base is a natural or non-natural nucleotide base;
L is a bond or a linker;
FG is a fluorogenic small molecule; and
R′ is hydrogen, or group comprising a fluorophore, fluorogenic small molecule, or quencher.

In certain embodiments, the fluorogenic reversible terminator NTP is capable of being deprotected under photochemical conditions. Therefore, in certain embodiments, the fluorogenic reversible terminator NTP is protected at the 2′-O— and/or 3′-O-positions with a photolabile oxygen protecting group. In certain embodiments, a fluorogenic reversible terminator NTP is protected at the 2′-O position with a photolabile protecting group. In certain embodiments, a fluorogenic reversible terminator NTP is protected at the 3′-O position with a photolabile protecting group.

In certain embodiments, a 2′- or 3′-O-protecting group (e.g., R^{P}) is of one of the following formulae:

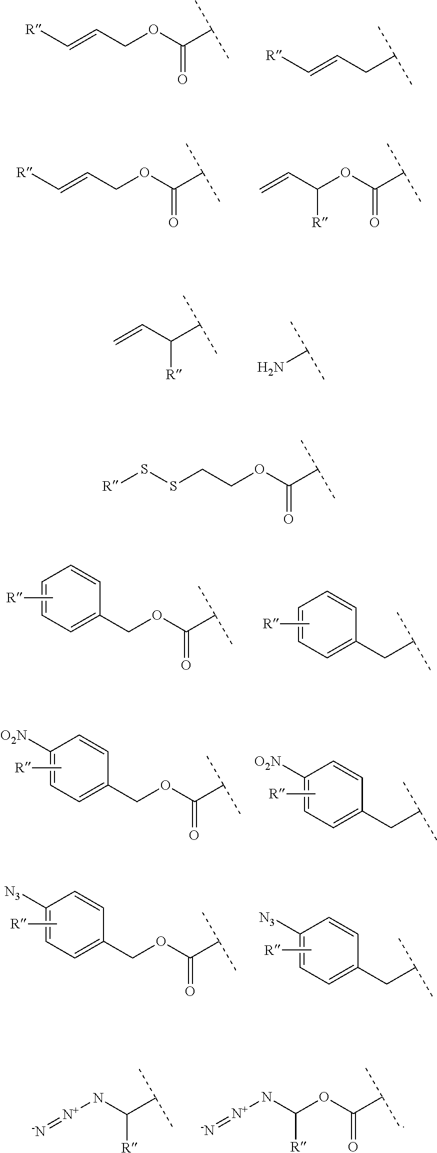

In certain embodiments, a 2′- or 3-O-protecting group (e.g., R^{P}) is of one of the following formulae:

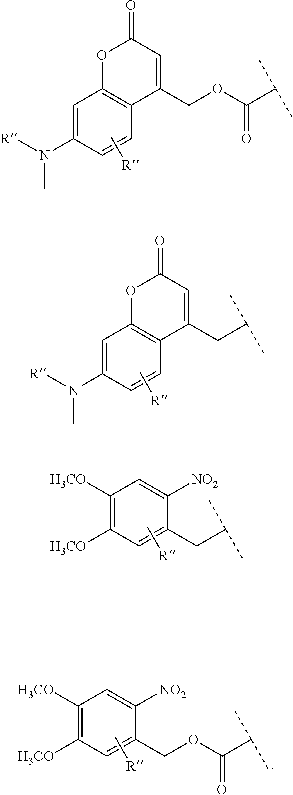

In certain embodiments, a 2′- or 3′-O-protecting group (e.g., R^{P}) is of one of the following formulae:

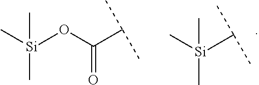

In certain embodiments, a 2′- or 3′-O-protecting group (e.g., R^{P}) is of the following formulae:

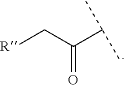

In certain embodiments, a 2′- or 3′-O-protecting group (e.g., R^{P}) is an amino acid of the following formula:

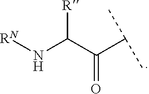

As defined herein, each R^{P} is independently an oxygen protecting group, optionally substituted acyl, or an amino acid. In certain embodiments, R^{P} is an oxygen protecting group. In certain embodiments, R^{P} is optionally substituted acyl. In certain embodiments, R^{P} is an amino acid. In certain embodiments, R^{P} is an oxygen protecting group, optionally substituted acyl, or an amino acid that can be cleaved by an esterase. In certain embodiments, each instance of R^{P} is independently alkyl, silyl, allyl, azidomethyl, benzyl, coumarinyl, or carbonate.

As defined herein, Y is O, S, or Se. In certain embodiments, Y is O. In certain embodiments, Y is S. In certain embodiments, Y is Se.

As defined herein, “Base” can be any natural or non-naturally occurring nucleobase. Naturally occurring bases include G, U, A, and C. Non-natural (e.g., modified) bases include substituted or modified variants of G, U, A, and C. Non-limiting examples of modified bases include, but are not limited to, 5-methylcytosine, pyridin-4-one, pyridin-2-one, phenyl, pseudouracil, 3-methyl uracil, dihydrouridine, naphthyl, aminophenyl, 5-alkylcytidines, 5-alkyluridines, 5-halouridines, 6-azapyrimidines, 6-alkylpyrimidines, propyne, quesosine, 2-thiouridine, 4-thiouridine, 4-acetyltidine, 5-(carboxyhydroxymethyl)uridine, 5-carboxymethylaminomethyl-2-thiouridine, 5-carboxymethylaminomethyluridine, β-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N₆-isopentenyladenosine, β-D-mannosylqueosine, uridine-5-oxyacetic acid, 2-thiocytidine, and threonine derivatives. Other non-limiting examples of bases include, but are not limited to, natural or non-natural pyrimidine or purine; and may include, but are not limited to, N¹-methyl-adenine, N⁶-methyl-adenine, 8′-azido-adenine, N,N-dimethyl-adenosine, aminoallyl-adenosine, 5′-methyl-urdine, pseudouridine, N¹-methyl-pseudouridine, 5′-hydroxy-methyl-uridine, 2′-thio-uridine, 4′-thio-uridine, hypoxanthine, xanthine, 5′-methyl-cytidine, 5′-hydroxy-methyl-cytidine, 6′-thio-guanine, and N⁷-methyl-guanine.

In certain embodiments, R is hydrogen, halogen, —CN, —NO₂, —N₃, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted hydroxyl, optionally substituted amino, or optionally substituted thiol. In certain embodiments, R is hydrogen. In certain embodiments, R is halogen. In certain embodiments, R is —CN. In certain embodiments, R is —NO₂. In certain embodiments, R is —N₃. In certain embodiments, R is optionally substituted alkyl. In certain embodiments, R is optionally substituted alkenyl. In certain embodiments, R is optionally substituted alkynyl. In certain embodiments, R is optionally substituted aryl. In certain embodiments, R is optionally substituted heteroaryl. In certain embodiments, R is optionally substituted carbocyclyl. In certain embodiments, R is optionally substituted heterocyclyl. In certain embodiments, R is optionally substituted acyl. In certain embodiments, R is optionally substituted hydroxyl. In certain embodiments, R is optionally substituted amino. In certain embodiments, R is optionally substituted thiol.

In certain embodiments, R is —OR^{P}, wherein R^{P} is an oxygen protecting group, optionally substituted acyl, or an amino acid.

In some embodiments, R is halogen, e.g., a fluorine group; an alkyl group, e.g., methyl or ethyl group; a methoxy group; an amino group; a thio group; an aminopropyl group; a dimethylaminoethyl; a dimethylaminopropyl group; a dimethylaminoethyloxyethyl group; an azido group; a silyl group; a cyclic alkyl group; or a N-methylacetamido group. In certain embodiments, R is hydroxyl (—OH), hydrogen (—H), fluoro (—F), amine (—NH₃), azido (—N₃), thiol (—SH), methoxy (—OCH₃), or methoxyethanol (—OCH₂CH₂OCH₃).

As generally defined herein, R′ is hydrogen, or a group comprising a fluorophore, fluorogenic small molecule, or fluorescent quencher. In certain embodiments, R′ is hydrogen. In certain embodiments, R′ is group comprising a fluorophore. In certain embodiments, R′ is a group comprising a fluorogenic small molecule. In certain embodiments, R′ is a group comprising fluorescent quencher.

As generally defined herein, R″ is hydrogen, halogen, —CN, —NO₂, —N₃, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted hydroxyl, optionally substituted amino, or optionally substituted thiol. In certain embodiments, R″ is hydrogen. In certain embodiments, R″ is halogen. In certain embodiments, R″ is —CN. In certain embodiments, R″ is —NO₂. In certain embodiments, R″ is —N₃. In certain embodiments, R″ is optionally substituted alkyl. In certain embodiments, R″ is optionally substituted alkenyl. In certain embodiments, R″ is optionally substituted alkynyl. In certain embodiments, R″ is optionally substituted aryl. In certain embodiments, R″ is optionally substituted heteroaryl. In certain embodiments, R″ is optionally substituted carbocyclyl. In certain embodiments, R″ is optionally substituted heterocyclyl. In certain embodiments, R″ is optionally substituted acyl. In certain embodiments, R″ is optionally substituted hydroxyl. In certain embodiments, R″ is optionally substituted amino. In certain embodiments, R″ is optionally substituted thiol.

As generally defined herein, R″′ is hydrogen, halogen, —CN, —NO₂, —N₃, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted hydroxyl, optionally substituted amino, optionally substituted thiol, or an oxygen protecting group. In certain embodiments, R″′ is hydrogen. In certain embodiments, R″′ is halogen. In certain embodiments, R″′ is —CN. In certain embodiments, R″′ is —NO₂. In certain embodiments, R″′ is —N₃. In certain embodiments, R″′ is optionally substituted alkyl. In certain embodiments, R″′ is optionally substituted alkenyl. In certain embodiments, R″′ is optionally substituted alkynyl. In certain embodiments, R″′ is optionally substituted aryl. In certain embodiments, R″′ is optionally substituted heteroaryl. In certain embodiments, R″′ is optionally substituted carbocyclyl. In certain embodiments, R″′ is optionally substituted heterocyclyl. In certain embodiments, R″′ is optionally substituted acyl. In certain embodiments, R″′ is optionally substituted hydroxyl. In certain embodiments, R″′ is optionally substituted amino. In certain embodiments, R″′ is optionally substituted thiol.

As generally defined herein, R^{N} is hydrogen, optionally substituted alkyl, optionally substituted acyl, or a nitrogen protecting group. In certain embodiments, R^{N} is hydrogen. In certain embodiments, R^{N} is optionally substituted alkyl. In certain embodiments, R^{N} is optionally substituted acyl. In certain embodiments, R^{N} is a nitrogen protecting group.

### Fluorogenic Small Molecules

Fluorogenic nucleosides (e.g., fluorogenic NTPs) described herein comprise fluorogenic small molecules. In certain embodiments, the fluorogenic small molecule comprises one of the following formulae:

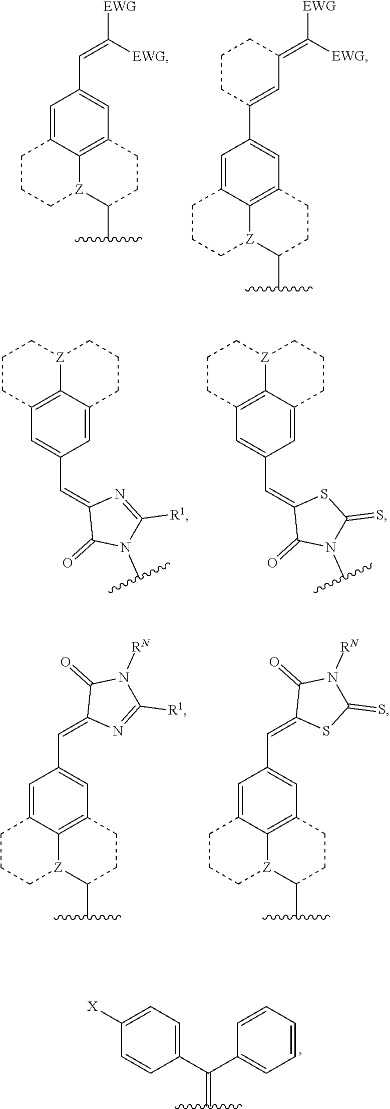

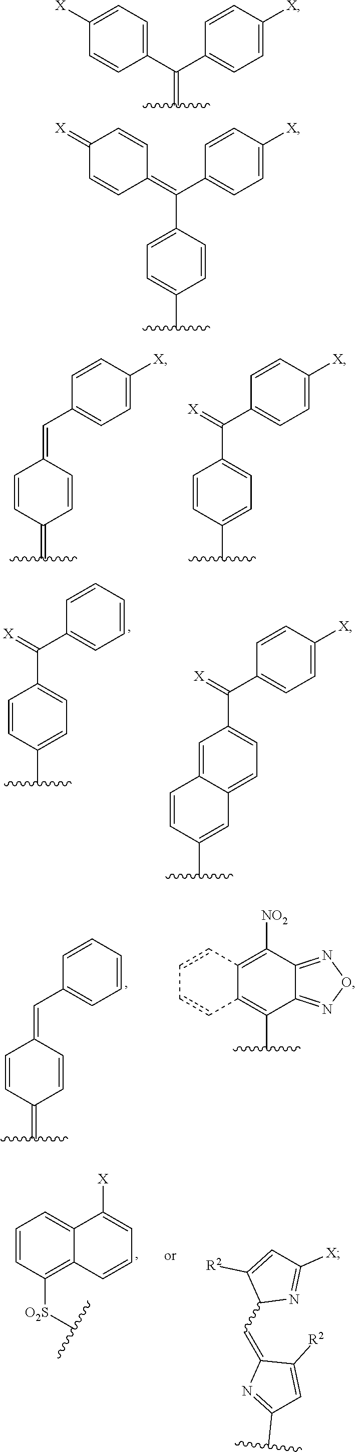

wherein:
each instance of EWG is independently an electron withdrawing group (e.g., —CN, optionally substituted acyl);
Z is N, NR^{N}, O, or S, as valency permits;
each instance of X is independently N(R^{N}), N(R^{N})₂, O, OR^{O}, S, or SR^{S}, as valency permits;
each instance of R¹ and R² is independently hydrogen, halogen, —CN, —NO₂, —N₃, —N(R^{N})₂, —OR^{O}, —SR^{S}, alkyl, alkenyl, alkynyl, carbocyclyl, aryl, heterocyclyl, heteroaryl, acyl, sulfinyl, or sulfonyl; and
each instance of R^{N}, R^{O}, and R^{S} is independently hydrogen, alkyl, alkenyl, alkynyl, carbocyclyl, aryl, heterocyclyl, heteroaryl, or acyl; and
wherein each formula is further optionally substituted.

For example, in certain embodiments, the fluorogenic small molecule comprises one of the following:

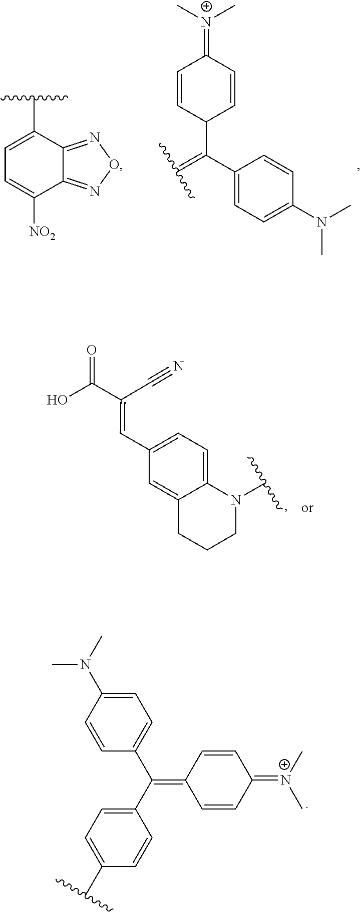

Other examples of fluorogenic moieties (e.g., fluorogenic small molecules) can be found in, e.g., International PCT Application Publication No. WO 2021/118727, published Jun. 17, 2021; and International PCT Application No. PCT/US2022/021878, filed Mar. 25, 2022.

In certain embodiments, the fluorogenic small molecule is conjugated to the nucleoside (e.g., NTP) via a bond. In certain embodiments, the fluorogenic small molecule is conjugated to the nucleoside (e.g., NTP) via a linker (e.g., a non-cleavable linker). As described herein, the bond or linker can be represented by the group L. In certain embodiments, L is a bond. In certain embodiments, L is a linker (e.g., a non-cleavable linker).

In certain embodiments, the linker (e.g., L) is selected from optionally substituted alkylene, optionally substituted heteroalkylene, optionally substituted alkenylene, optionally substituted alkynylene, optionally substituted acylene, optionally substituted carbocyclylene, optionally substituted heterocyclylene, optionally substituted arylene, optionally substituted heteroarylene, and any combination thereof. In certain embodiments, the linker (e.g., L) comprises optionally substituted alkylene. In certain embodiments, the linker (e.g., L) comprises optionally substituted heteroalkylene. In certain embodiments, the linker (e.g., L) comprises optionally substituted alkenylene. In certain embodiments, the linker (e.g., L) comprises optionally substituted alkynylene. In certain embodiments, the linker (e.g., L) comprises optionally substituted acylene. In certain embodiments, the linker (e.g., L) comprises optionally substituted carbocyclylene. In certain embodiments, the linker (e.g., L) comprises optionally substituted heterocyclylene. In certain embodiments, the linker (e.g., L) comprises optionally substituted arylene. In certain embodiments, the linker (e.g., L) comprises optionally substituted heteroarylene.

In certain embodiments, a fluorogenic NTP provided herein is selected from:

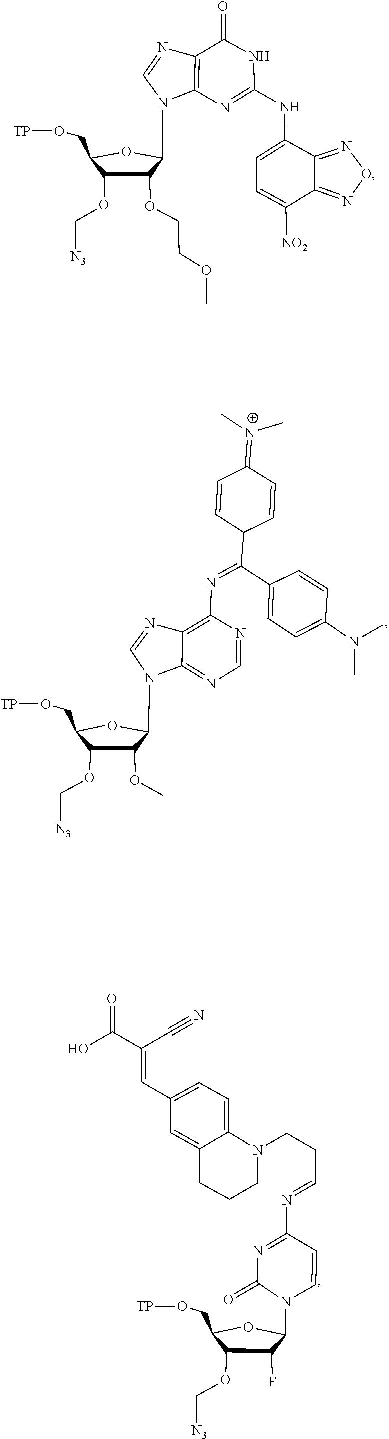

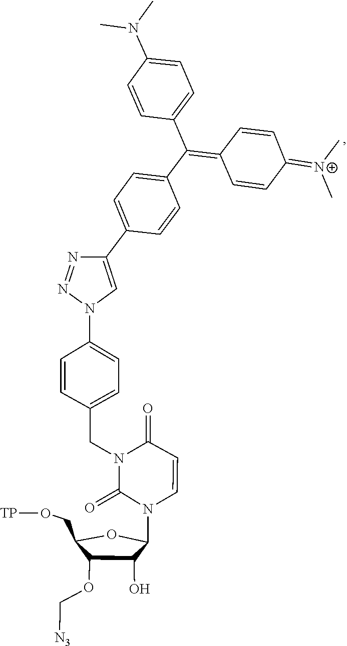

and salts and tautomers thereof, wherein TP is a triphosphate group. In certain embodiments, TP can be hydrogen, or a mono-, di-, or triphosphate group.

### Fluorogenic Oligonucleotide Synthesis

The fluorogenic NTPs provided herein can be used in the chemical or enzymatic synthesis of oligonucleotides (e.g., DNA or RNA oligonucleotides, DNA or RNA aptamers).

In one aspect, provided herein are methods for the synthesis of oligonucleotides (e.g., RNA or DNA oligonucleotides, e.g., RNA aptamers) wherein a polymerase (e.g., poly(N) polymerase, such as a poly(U) polymerase) incorporates one or more fluorogenic NTPs onto an initiator oligonucleotide via a terminal transferase. In certain embodiments, provided herein are methods for the template-independent synthesis of an RNA oligonucleotide (e.g., an RNA aptamer), the method comprising:
(a) providing an initiator oligonucleotide, wherein the initiator oligonucleotide is single-stranded RNA;
(b) providing a polymerase; and
(c) combining the initiator oligonucleotide, the polymerase, and a fluorogenic NTP described herein (e.g., a fluorogenic 2′- and/or 3′-reversible terminator NTP) under conditions sufficient for the addition of the NTP to the 3′ end of the initiator oligonucleotide.

In certain embodiments, when the fluorogenic NTP is a fluorogenic 2′- and/or 3′-reversible terminator NTP, the method further comprises a step of:
(d) deprotecting the 2′- and/or 3′-reversible terminator group at the 3′ end of the oligonucleotide formed in step (c).

In certain embodiments, the method further comprises a step of:
(e) incorporating one or more nucleoside triphosphates to the 3′ end of the RNA oligonucleotide formed in step (d).

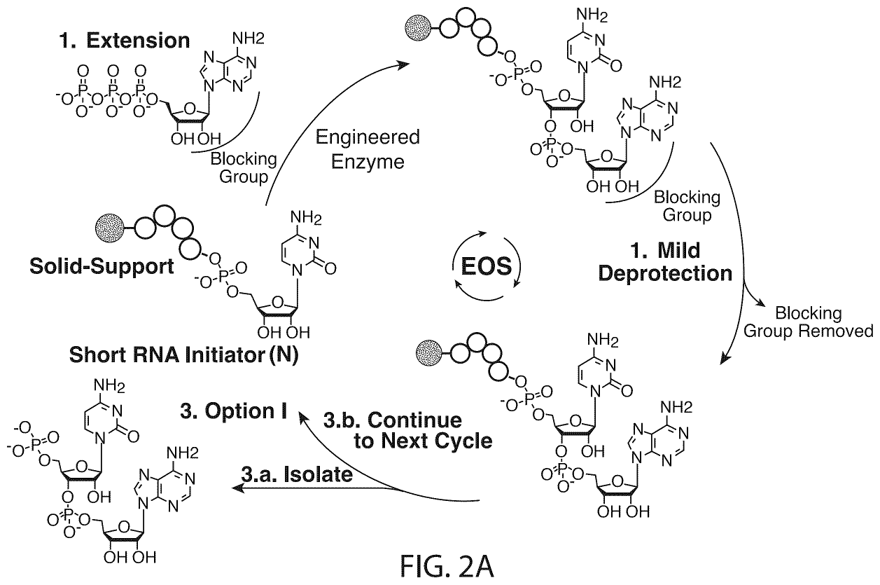

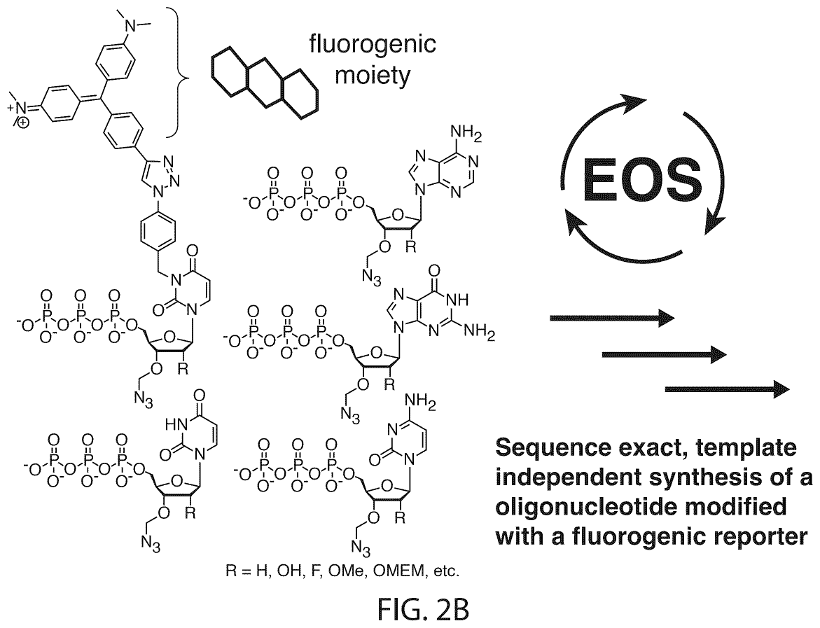

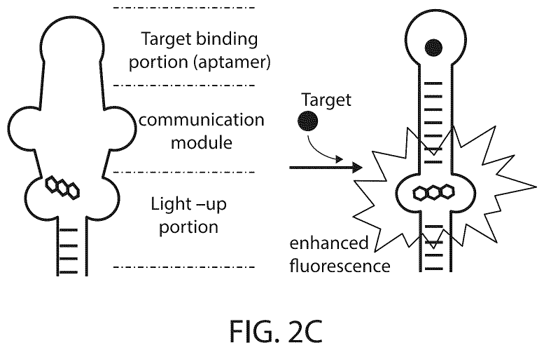

Use of fluorogenic reversible terminator NTPs (e.g., 2′-O— or 3′-O-protected reversible terminator NTPs) allow (n+1) incorporation of the fluorogenic NTP. Incorporation of a reversible terminator NTP (e.g., a 2′-O— or 3′-O-protected reversible terminator NTPs) reversibly alters the binding affinity of the polymerase (e.g., a poly(N) polymerase, such as poly(U) polymerase) to the extended initiator oligonucleotide. This change in binding affinity results in the termination of further nucleotide addition, producing an (n+1) oligonucleotide product that can be further extended after the modified group is restored to its natural state (e.g., a 2′- or 3′—OH group) via mild deprotection chemistry. An “(n+1) oligonucleotide” is a product wherein a single nucleotide has been added to the initiator sequence. These methods are exemplified in the generic schemes shown in FIGS. 2A-2C.

In certain embodiments, the polymerase used in the methods described herein is a poly(N) polymerase. Examples of polymerases are provided herein, including but not limited to the following. Other materials and methods for incorporating the fluorogenic nucleosides (e.g., fluorogenic NTPs) provided herein, including other polymerases, are described in, e.g., International PCT Application Publication No. WO 2020/077227, published Apr. 16, 2020, the entire contents of which is incorporated herein by reference.

### Poly(N) Polymerases

The enzyme for use in incorporating one or more fluorogenic NTPs can be an RNA polymerase, such as a poly(N) polymerase. Provided herein are poly(N) polymerases, e.g., mutant (i.e., mutated) poly(U) polymerases, that are useful in the methods described herein.

In certain embodiments, the poly(N) polymerase is a poly(U) polymerase, a poly(A) polymerase, a poly(C) polymerase, or a poly(G) polymerase. The RNA polymerase may be a wild-type polymerase, or a mutant (i.e., mutated), variant, or homolog thereof. In certain embodiments, the poly(N) polymerase is a wild-type polymerase. In certain embodiments, the polymerase is a mutant of a poly(N) polymerase. In certain embodiments, the polymerase is a variant of a poly(N) polymerase. In certain embodiments, a mutant or variant is approximately 60%, 65%, 70%, 75%, 80%, 85%, 90%, 95%, 98%, or 99% identical to the wild-type polymerase. In certain embodiments, the polymerase is a homolog of a poly(N) polymerase.

In certain embodiments, the poly(N) polymerase is a poly(U) polymerase. In certain embodiments, the poly(U) polymerase is wild-type Schizosaccharomyces pombe poly(U) polymerase, or a mutant thereof, or a homolog thereof. In certain embodiments, the poly(U) polymerase is wild-type Schizosaccharomyces pombe poly(U) polymerase. In certain embodiments, the poly(U) polymerase is a mutant of Schizosaccharomyces pombe poly(U) polymerase. In certain embodiments, the poly(U) polymerase is a variant of Schizosaccharomyces pombe poly(U) polymerase. In certain embodiments, the poly(U) polymerase is a homolog of Schizosaccharomyces pombe poly(U) polymerase.

In certain embodiments, the poly(N) polymerase is a poly(A) polymerase. In certain embodiments, the poly(A) polymerase is wild-type Saccharomyces cerevisiae poly(A) polymerase, or a mutant thereof. In certain embodiments, the poly(N) polymerase is wild-type Saccharomyces cerevisiae poly(A) polymerase. In certain embodiments, the poly(N) polymerase is a mutant of Saccharomyces cerevisiae poly(A) polymerase. In certain embodiments, the poly(N) polymerase is a variant of Saccharomyces cerevisiae poly(A) polymerase. In certain embodiments, the poly(N) polymerase is a homolog of Saccharomyces cerevisiae poly(A) polymerase.

### Poly(U) Polymerase Mutants

As described herein, in certain embodiments, the poly(N) polymerase is a mutant of a poly(N) polymerase (i.e., mutated poly(N) polymerase). In certain embodiments, the poly(N) polymerase is a Schizosaccharomyces pombe poly(U) (S. pombe poly(U)) polymerase comprising mutations at one or more positions selected from H336, N171, and T172.

In certain embodiments, the poly(N) polymerase is a Schizosaccharomyces pombe poly(U) (S. pombe poly(U)) polymerase comprising an H336 mutation (i.e., wherein the amino acid H at position 336 is replaced with another amino acid). In certain embodiments, the poly(N) polymerase is an S. pombe poly(U) polymerase comprising an H336 mutation selected from the group consisting of H336A H336C, H336D, H336E, H336F, H336G, H336I, H336K, H336L, H336M, H336T, H336V, H336W, H336Y, H336N, H336P, H336Q, H336R, H336S, and H336W. In certain embodiments, the H336 mutation is the only mutation. In certain embodiments, the poly(N) polymerase comprises one or more addition mutations and is about 60%, 65%, 70%, 75%, 80%, 85%, 90%, 95%, 98%, or 99% identical to SEQ ID NO: 3.

Schizosaccharomyces pombe Poly(U) Polymerase:

(SEQ ID NO: 3)
MNISSAQFIPGVHTVEEIEAEIHKNLHISKSCSYQKVPNSHKEFTKFCYE
VYNEIKISDKEFKEKRAALDTLRLCLKRISPDAELVAFGSLESGLALKNS
DMDLCVLMDSRVQSDTIALQFYEELIAEGFEGKFLQRARIPIIKLTSDTK
NGFGASFQCDIGFNNRLAIHNTLLLSSYTKLDARLKPMVLLVKHWAKRKQ
INSPYFGTLSSYGYVLMVLYYLIHVIKPPVFPNLLLSPLKQEKIVDGFDV
GFDDKLEDIPPSQNYSSLGSLLHGFFRFYAYKFEPREKVVTFRRPDGYLT
KQEKGWTSATEHTGSADQIIKDRYILAIEDPFEISHNVGRTVSSSGLYRI
RGEFMAASRLLNSRSYPIPYDSLFEEAPIPPRROKKTDEQSNKKLLNETD
GDNSE

In certain embodiments, the poly(N) polymerase is identical to SEQ ID NO: 3, but includes one H336 mutation selected from the group consisting of H336A, H336C, H336D, H336E, H336F, H336G, H336I, H336K, H336L, H336M, H336T, H336V, H336W, H336Y, H336N, H336P, H336Q, H336R, H336S, and H336W.

In certain embodiments, the poly(N) polymerase is an S. pombe poly(U) polymerase comprising an H336R mutation. In certain embodiments, the H336R mutation is the only mutation. In certain embodiments, the poly(N) polymerase comprises one or more addition mutations and is about 60%, 65%, 70%, 75%, 80%, 85%, 90%, 95%, 98%, or 99% identical to SEQ ID NO: 3. In certain embodiments, the poly(N) polymerase is identical to SEQ ID NO: 3, but includes one mutation: H336R.

In certain embodiments, the poly(N) polymerase is an S. pombe poly(U) polymerase comprising an N171 mutation. In certain embodiments, the poly(N) polymerase is an S. pombe poly(U) polymerase comprising an N171 mutation selected from the group consisting of N171E, N171L, N171Q, N171S, N171M, N171D, N171G, N171C, N171A, N171W, N171T, N171I, N171V, N171P, N171R, N171H, and N171K. In certain embodiments, the N171 mutation is the only mutation. In certain embodiments, the poly(N) polymerase comprises one or more additional mutations and is about 60%, 65%, 70%, 75%, 80%, 85%, 90%, 95%, 98%, or 99% identical to SEQ ID NO: 3. In certain embodiments, the poly(N) polymerase is identical to SEQ ID NO: 3, but includes one N171 mutation selected from the group consisting of N171E, N171L, N171Q, N171S, N171M, N171D, N171G, N171C, N171A, N171W, N171T, N171I, N171V, N171P, N171R, N171H, and N171K.

In certain embodiments, the poly(N) polymerase is an S. pombe poly(u) polymerase comprising an N171A mutation. In certain embodiments, the N171A mutation is the only mutation. In certain embodiments, the poly(N) polymerase comprises one or more addition mutations and is about 60%, 65%, 70%, 75%, 80%, 85%, 90%, 95%, 98%, or 99% identical to SEQ ID NO: 3. In certain embodiments, the poly(N) polymerase is identical to SEQ ID NO: 3, but includes one mutation: N171A.

In certain embodiments, the poly(N) polymerase is an S. pombe poly(U) polymerase comprising an N171T mutation. In certain embodiments, the N171T mutation is the only mutation. In certain embodiments, the poly(N) polymerase comprises one or more addition mutations and is about 60%, 65%, 70%, 75%, 80%, 85%, 90%, 95%, 98%, or 99% identical to SEQ ID NO: 3. In certain embodiments, the poly(N) polymerase is identical to SEQ ID NO: 3, but includes one mutation: N171T.

In certain embodiments, the poly(N) polymerase is an S. pombe poly(U) polymerase comprising an T172 mutation. In certain embodiments, the poly(N) polymerase is an S. pombe poly(U) polymerase comprising an T172 mutation selected from the group consisting of T172E, T172L, T172Q, T172S, T172M, T172D, T172G, T172C, T172A, T172W, T172T, T172I, T172V, T172P, T172R, T172H, and T172K. In certain embodiments, the T172 mutation is the only mutation. In certain embodiments, the poly(N) polymerase comprises one or more addition mutations and is about 60%, 65%, 70%, 75%, 80%, 85%, 90%, 95%, 98%, or 99% identical to SEQ ID NO: 3. In certain embodiments, the poly(N) polymerase is identical to SEQ ID NO: 3, but includes one T172 mutation selected from the group consisting of T172E, T172L, T172Q, T172S, T172M, T172D, T172G, T172C, T172A, T172W, T172T, T172I, T172V, T172P, T172R, T172H, and T172K.

In certain embodiments, the poly(N) polymerase is an S. pombe poly(U) polymerase comprising H336 and N171 mutations. In certain embodiments, the poly(N) polymerase is an S. pombe poly(U) polymerase comprising an H336 mutation selected from the group consisting of H336A H336C, H336D, H336E, H336F, H336G, H336I, H336K, H336L, H336M, H336T, H336V, H336W, H336Y, H336N, H336P, H336Q, H336R, H336S, and H336W; and an N171 mutation selected from the group consisting of N171E, N171L, N171Q, N171S, N171M, N171D, N171G, N171C, N171A, N171W, N171T, N171I, N171V, N171P, N171R, N171H, and N171K. In certain embodiments, the H336 and N171 mutations are the only mutations. In certain embodiments, the poly(N) polymerase comprises one or more addition mutations and is about 60%, 65%, 70%, 75%, 80%, 85%, 90%, 95%, 98%, or 99% identical to SEQ ID NO: 3. In certain embodiments, the poly(N) polymerase is identical to SEQ ID NO: 3, but includes one H336 mutation selected from the group consisting of H336A H336C, H336D, H336E, H336F, H336G, H336I, H336K, H336L, H336M, H336T, H336V, H336W, H336Y, H336N, H336P, H336Q, H336R, H336S, and H336W; and one N171 mutation selected from the group consisting of N171E, N171L, N171Q, N171S, N171M, N171D, N171G, N171C, N171A, N171W, N171T, N171I, N171V, N171P, N171R, N171H, and N171K.

In certain embodiments, the poly(N) polymerase is an S. pombe poly(U) polymerase comprising H336R and N171A mutations. In certain embodiments, the H336R and N171A mutations are the only mutations. In certain embodiments, the poly(N) polymerase comprises one or more addition mutations and is about 60%, 65%, 70%, 75%, 80%, 85%, 90%, 95%, 98%, or 99% identical to SEQ ID NO: 3. In certain embodiments, the poly(N) polymerase is identical to SEQ ID NO: 3, but includes two mutations: H336R and N171A.

In certain embodiments, the poly(N) polymerase is an S. pombe poly(U) polymerase comprising H336R and N171T mutations. In certain embodiments, the H336R and N171T mutations are the only mutations. In certain embodiments, the poly(N) polymerase comprises one or more addition mutations and is about 60%, 65%, 70%, 75%, 80%, 85%, 90%, 95%, 98%, or 99% identical to SEQ ID NO: 3. In certain embodiments, the poly(N) polymerase is identical to SEQ ID NO: 3, but includes two mutations: H336R and N171T.

In certain embodiments, the poly(N) polymerase is an S. pombe poly(U) polymerase comprising H336 and T172 mutations. In certain embodiments, the poly(N) polymerase is an S. pombe poly(U) polymerase comprising an H336 mutation selected from the group consisting of H336A H336C, H336D, H336E, H336F, H336G, H336I, H336K, H336L, H336M, H336T, H336V, H336W, H336Y, H336N, H336P, H336Q, H336R, H336S, and H336W; and a T172 mutation selected from the group consisting of T172E, T172L, T172Q, T172S, T172M, T172D, T172G, T172C, T172A, T172W, T172T, T172I, T172V, T172P, T172R, T172H, and T172K. In certain embodiments, the H336 and T172 mutations are the only mutations. In certain embodiments, the poly(N) polymerase comprises one or more addition mutations and is about 60%, 65%, 70%, 75%, 80%, 85%, 90%, 95%, 98%, or 99% identical to SEQ ID NO: 3. In certain embodiments, the poly(N) polymerase is identical to SEQ ID NO: 3, but includes one H336 mutation selected from the group consisting of H336A H336C, H336D, H336E, H336F, H336G, H336I, H336K, H336L, H336M, H336T, H336V, H336W, H336Y, H336N, H336P, H336Q, H336R, H336S, and H336W; and one T172 mutation selected from the group consisting of T172E, T172L, T172Q, T172S, T172M, T172D, T172G, T172C, T172A, T172W, T172T, T172I, T172V, T172P, T172R, T172H, and T172K. In certain embodiments, the H336 mutation is H336R.

In certain embodiments, the poly(N) polymerase is an S. pombe poly(U) polymerase comprising H336, N171, and T172 mutations. In certain embodiments, the poly(N) polymerase is an S. pombe poly(U) polymerase comprising an H336 mutation selected from the group consisting of H336A H336C, H336D, H336E, H336F, H336G, H336I, H336K, H336L, H336M, H336T, H336V, H336W, H336Y, H336N, H336P, H336Q, H336R, H336S, and H336W; an N171 mutation selected from the group consisting of N171E, N171L, N171Q, N171S, N171M, N171D, N171G, N171C, N171A, N171W, N171T, N171I, N171V, N171P, N171R, N171H, and N171K; and a T172 mutation selected from the group consisting of T172E, T172L, T172Q, T172S, T172M, T172D, T172G, T172C, T172A, T172W, T172T, T172I, T172V, T172P, T172R, T172H, and T172K. In certain embodiments, the H336, N171, and T172 mutations are the only mutations. In certain embodiments, the poly(N) polymerase comprises one or more addition mutations and is about 60%, 65%, 70%, 75%, 80%, 85%, 90%, 95%, 98%, or 99% identical to SEQ ID NO: 3. In certain embodiments, the poly(N) polymerase is identical to SEQ ID NO: 3, but includes one H336 mutation selected from the group consisting of H336A H336C, H336D, H336E, H336F, H336G, H336I, H336K, H336L, H336M, H336T, H336V, H336W, H336Y, H336N, H336P, H336Q, H336R, H336S, and H336W; one N171 mutation selected from the group consisting of N171E, N171L, N171Q, N171S, N171M, N171D, N171G, N171C, N171A, N171W, N171T, N171I, N171V, N171P, N171R, N171H, and N171K; and one T172 mutation selected from the group consisting of T172E, T172L, T172Q, T172S, T172M, T172D, T172G, T172C, T172A, T172W, T172T, T172I, T172V, T172P, T172R, T172H, and T172K. In certain embodiments, the H336 mutation is H336R. In certain embodiments, the N171 mutation is N171A or N171T.

### Fluorogenic Oligonucleotide Synthesis Reactions

The terminal transferase reactions described herein (i.e., step (c) of any of the methods described herein) are carried out in the presence of a polymerase enzyme (e.g., a poly(N) polymerase). In certain embodiments, step (c) is carried out in the presence of one or more additional enzymes. In certain embodiments, step (c) is carried out in the presence of a mixture of two or more different enzymes. The mixture of enzymes may comprise more than one distinct poly(N) polymerases (e.g., 2 or 3 different poly(N) polymerases). The mixture of poly(N) polymerase enzymes may include both wild-type and mutates poly(N) polymerases (e.g., mutated poly(U) polymerases provided herein).

In certain embodiments, step (c) is carried out in the presence of one or more additional phosphatases in addition to the poly(N) polymerase. In certain embodiments, step (c) is carried out in the presence of a yeast inorganic pyrophosphatase (PPI-ase) in addition to the poly(N) polymerase.

In certain embodiments, the terminal transferase reaction in step (c) is carried out in the presence of one or more additional additives. In certain embodiments, step (c) is carried out in the presence of a crowding agent. In certain embodiments, the crowing agent is polyethylene glycol (PEG) or Ficoll. In certain embodiments, the crowding agent is polyethylene glycol (PEG). In certain embodiments, step (c) is carried out in the presence of an RNase inhibitor. In certain embodiments, step (c) is carried out in the presence of a non-hydrolyzable nucleotide.

### Initiator Oligonucleotides

Some methods described herein use initiator oligonucleotides. The initiator oligonucleotides may be of any sequence and can be any number of nucleotides in length. In certain embodiments, the initiator oligonucleotide is 20 nucleotides or less in length. In certain embodiments, the initiator oligonucleotide is 5-20 nucleotides in length. In certain embodiments, the initiator oligonucleotide is more than 20 nucleotides in length.

In certain embodiments, the initiator oligonucleotide is a poly-rN oligonucleotide. In certain embodiments, the initiator oligonucleotide is a poly-rU, poly-rC, poly-rG, or poly-rA.

The initiator oligonucleotide may also be covalently linked to a solid support. In certain embodiments, the oligonucleotide is cleaved from the solid support after a desired RNA oligonucleotide sequence is obtained. Therefore, in certain embodiments, the initiator oligonucleotide is covalently linked to a solid support through a cleavable linker.

The initiator oligonucleotides can comprise other modification such as fluorophores. In certain embodiments, the initiator oligonucleotide comprises a 5′-fluorophore. In certain embodiments, the fluorophore is Cy5 or FAM. The initiator oligonucleotide may also comprise one or more additional functional groups or handles for bioconjugation. In certain embodiments, the initiator oligonucleotide is functionalized with biotin.

In certain embodiments, the initiator oligonucleotide comprises a 5′-phosphate (e.g., 5′-mono-, di-, or triphosphate). In certain embodiments, the initiator oligonucleotide comprises a 5′-monophosphate. In certain embodiments, the initiator oligonucleotide comprises a 5′-diphosphate. In certain embodiments, the initiator oligonucleotide comprises a 5′-triphosphate.

In certain embodiments, the initiator oligonucleotide comprises a 5′-capping group (i.e., 5′ cap).

In certain embodiments, the 5′ cap can be a mono-nucleotide (1-nt), di-nucleotide (2-nt), tri-nucleotide (3-nt), or N-nucleotide (i.e., of any oligonucleotide length that would be useful). The 5′ cap may also comprise a combination of one or more natural and/or non-natural (e.g., modified) nucleoside bases, including those described herein.

In certain embodiments, the 5′ cap is a guanine cap. In certain embodiments, the 5′ cap is a 7-methylguanylate cap (m⁷G). A In certain embodiments, the guanine or m⁷G cap includes a guanine nucleotide connected to the oligonucleotide via a 5′ to 5′ triphosphate linkage. In certain embodiments, the 5′ cap includes methylation of the 2′ hydroxy-groups of the first and/or second 2 ribose sugars of the 5′ end of the oligonucleotide.

In certain embodiments, the 5′-cap is a 5′-trimethylguanosine cap or a 5′-monomethylphosphate cap. In other embodiments, the 5′ cap is a NAD⁺, NADH, or 3′-dephospho-coenzyme A cap.

In certain embodiments, the initiator oligonucleotide comprises a primer site for reverse transcription of the synthesized RNA oligonucleotide. In certain embodiments, the initiator oligonucleotide comprises a primer site for PCR amplification.

### Fluorogenic Oligonucleotides and Methods

Provided herein are oligonucleotides comprising one or more fluorogenic NTPs described herein (i.e., “fluorogenic oligonucleotides”). In certain embodiments, the fluorogenic oligonucleotide is prepared by a method of the present disclosure. In certain embodiments, the fluorogenic oligonucleotide comprises one (1) fluorogenic NTP described herein. In certain embodiments, the fluorogenic oligonucleotide comprises more than one (e.g., 2, 3, 4, 5, or more) fluorogenic NTP described herein.

In certain embodiments, the fluorogenic oligonucleotide is an RNA oligonucleotide. In certain embodiments, the fluorogenic oligonucleotide is a DNA oligonucleotide. In certain embodiments, the fluorogenic oligonucleotide is an RNA aptamer (i.e., “fluorogenic RNA aptamer”).

As used herein, “aptamer” refers to a single-stranded oligonucleotide capable of folding into a defined architecture and binding to a target (e.g., protein). In certain embodiments, the fluorogenic RNA aptamers are “light-up” aptamers which, upon contacting a target, the RNA aptamer rigidifies, resulting in a change (e.g., increase) in fluorescence and/or fluorescence lifetime.

Provided herein are methods of detecting a target comprising:
(i) contacting a target with a fluorogenic oligonucleotide (e.g., a fluorogenic RNA aptamer) described herein; and
(ii) measuring or observing the fluorescence of the fluorogenic oligonucleotide (e.g., a fluorogenic RNA aptamer) and/or measuring or observing a change in the fluorescence lifetime of the fluorogenic oligonucleotide (e.g., a fluorogenic RNA aptamer).

Examples of targets detectable by the methods provided herein include, but are not limited to, proteins, biomarkers, pathogens, drugs, drug conjugates, nucleic acids, small molecules, and metabolites.

In certain embodiments of the methods provided herein, a change in fluorescence and/or fluorescence lifetime (e.g., an increase in fluorescence and/or fluorescence lifetime) is observed instantaneously after the contacting step. In certain embodiments, the change in fluorescence and/or fluorescence lifetime (e.g., increase in fluorescence and/or fluorescence lifetime) is observed within less than 1 second after the contacting step. In certain embodiments, the change in fluorescence and/or fluorescence lifetime (e.g., increase in fluorescence and/or fluorescence lifetime) is observed within less than 2500, 2000, 1500, 1000, 750, 500, or 250 milliseconds (ms) after the contacting step. In certain embodiments, the change is an increase in fluorescence and/or fluorescent lifetime. In certain embodiments, an increase in fluorescence of at least 1-fold, 2-fold, 5-fold, 10-fold, 20-fold, 25-fold, 30-fold, 35-fold, 40-fold, 45-fold, 50-fold, 100-fold, 150-fold, 200-fold, 300-fold, 400-fold, or 500-fold is observed.

In some embodiments, the methods of detecting provided herein can be used to observe the absorption, distribution, metabolism, and/or excretion (ADME) of therapeutic agents (e.g., drugs) in vivo. The methods of detecting provided herein can also be used for in vitro applications including, but not limited to, lateral flow assays. In a lateral flow assay, in certain embodiments, a fluorogenic RNA aptamer provided herein is immobilized on a solid support (e.g., a plate) and liquid containing the target substance is flowed over the solid support (e.g., plate).

Also provided herein are kits comprising one or more fluorogenic NTPs and/or one or more fluorogenic oligonucleotides (e.g., fluorogenic RNA aptamers) provided herein. The kits are useful in any one of the methods described herein (e.g., methods of preparing RNA oligonucleotides, methods of detecting a target).

### EXAMPLES

Fluorogenic NTPs described herein can be incorporated into oligonucleotides using materials and methods described in, e.g., International PCT Application Publication No. WO 2020/077227, published Apr. 16, 2020, the entire contents of which is incorporated herein by reference.

As shown in FIGS. 2A-2C, the fluorogenic NTPs template-independent, enzymatic oligonucleotide synthesis to prepare a “light up” oligonucleotide that is modified with a fluorogenic small molecule. FIG. 2A shows an overview of enzymatic oligonucleotide synthesis (EOS). One cycle consists of an extension (the addition of a reversible terminator NTP) followed by a mild deblocking. FIG. 2B shows examples of building blocks used in EOS to prepare “light-up” aptamers. Upon the introduction of the target, the oligonucleotide rigidifies resulting in an enhancment of fluorescence (FIG. 2C). See, e.g., Ryckelynck et al., Int. J. Mol. Sci. 2018, 19(1), 44.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein.

It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A nucleoside triphosphate (NTP) comprising a fluorogenic small molecule.

2. The NTP of claim 1, wherein the NTP comprises a ribose nucleoside.

3. The NTP of claim 1 or 2, wherein the fluorogenic small molecule is conjugated to the base, sugar, or triphosphate moiety of the NTP.

4. The NTP of any one of claims 1-3, wherein the fluorogenic small molecule is conjugated to the base moiety of the NTP.

5. The NTP of any one of claims 1-4, wherein the fluorogenic small molecule is conjugated to the NTP via a bond or a non-cleavable linker.

6. The NTP of claim 5, wherein the fluorogenic small molecule is conjugated to the NTP via a bond.

7. The NTP of claim 5, wherein the fluorogenic small molecule is conjugated to the NTP via a non-cleavable linker.

8. The NTP of any one of the preceding claims, wherein the NTP comprises a 2′-modification selected from halogen, optionally substituted alkyl, and optionally substituted hydroxyl.

9. The NTP of any one of claims 1-7 further comprising a 2′- and/or 3′-reversible terminator group.

10. The NTP of any one of the preceding claims, wherein the NTP comprises a 2′-reversible terminator group.

11. The NTP of any one of the preceding claims, wherein the NTP comprises a 2′-O-protected reversible terminator group.

12. The NTP of claim 9, wherein the NTP comprises a 2′-O-alkyl, 2′-O-silyl, 2′-O-allyl, 2′-O-azidomethyl, 2′-O-benzyl, 2′-O-coumarinyl, or 2′-O-carbonate group.

13. The NTP of claim 9, wherein the NTP comprises a 2′-O-carbonate group selected from 2′-O-allyloxycarbonyl and 2′-O-(2-oxo-2H-chromen-4-yl)methyloxycarbonyl.

14. The NTP of claim 9, wherein the NTP comprises a 2′-O-allyl, 2′-O-azidomethyl, 2′-O-allyl carbonate, 2′-O-azidomethyl carbonate, or 2′-azidoethoxy group.

15. The NTP of any one of the preceding claims, wherein the NTP comprises a 3′-reversible terminator group.

16. The NTP of any one of the preceding claims, wherein the NTP comprises a 3′-O-protected reversible terminator group.

17. The NTP of claim 14, wherein the NTP comprises a 3′-O-alkyl, 3′-O-silyl, 3′-O-allyl, 3′-O-azidomethyl, 3′-O-benzyl, 3′-O-coumarinyl, or 3′-O-carbonate group.

18. The NTP of claim 14, wherein the NTP comprises a 3′-O-carbonate group selected from 3′-O-allyloxycarbonyl and 3′-O-(2-oxo-2H-chromen-4-yl)methyloxycarbonyl.

19. The NTP of claim 14, wherein the NTP comprises a 3′-O-allyl, 3′-O-azidomethyl, 3′-O-allyl carbonate, 3′-O-azidomethyl carbonate, or 3′-azidoethoxy group.

20. The NTP of any one of the preceding claims, wherein the NTP comprises a modified base moiety.

21. The NTP of any one of the preceding claims, wherein the NTP comprises a modified triphosphate moiety.

22. The NTP of any one of the preceding claims, wherein the NTP is of the formula: 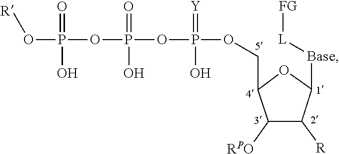
or a salt thereof, wherein:
Y is O, S, or Se;
R^{P} is an oxygen protecting group, optionally substituted acyl, or an amino acid;
R is hydrogen, halogen, —CN, —NO₂, —N₃, optionally substituted alkyl, optionally substituted alkenyl, optionally substituted alkynyl, optionally substituted aryl, optionally substituted heteroaryl, optionally substituted carbocyclyl, optionally substituted heterocyclyl, optionally substituted acyl, optionally substituted hydroxyl, optionally substituted amino, or optionally substituted thiol;
Base is a natural or non-natural nucleotide base;
L is a bond or a linker;
FG is a fluorogenic small molecule; and
R′ is hydrogen, or group comprising a fluorophore, fluorogenic small molecule, or fluorescent quencher.

23. The NTP of any one of the preceding claims, wherein the NTP is of the formula: 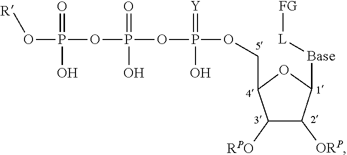
or a salt thereof, wherein:
Y is O, S, or Se;
each instance of R^{P} is hydrogen, an oxygen protecting group, optionally substituted acyl, or an amino acid, or two R^{P} are joined together with the intervening atoms to form optionally substituted heterocyclyl; provided that at least one R^{P} is an oxygen protecting group, optionally substituted acyl, or an amino acid; and
Base is a natural or non-natural nucleotide base;
L is a bond or a linker;
FG is a fluorogenic small molecule; and
R′ is hydrogen, or group comprising a fluorophore, fluorogenic small molecule, or quencher.

24. The NTP of claim 22 or 23, wherein Y is O.

25. The NTP of any one of claims 22-24, wherein R′ is hydrogen.

26. The NTP of any one of claims 22-25, wherein L is a bond.

27. The NTP of any one of claims 22-26, wherein L is a linker.

28. The NTP of any one of the preceding claims, wherein the linker is selected from optionally substituted alkylene, optionally substituted heteroalkylene, optionally substituted alkenylene, optionally substituted alkynylene, optionally substituted acylene, optionally substituted carbocyclylene, optionally substituted heterocyclylene, optionally substituted arylene, optionally substituted heteroarylene, and any combination thereof.

29. The NTP of any one of the preceding claims, wherein the fluorogenic small molecule comprises one of the following formulae: 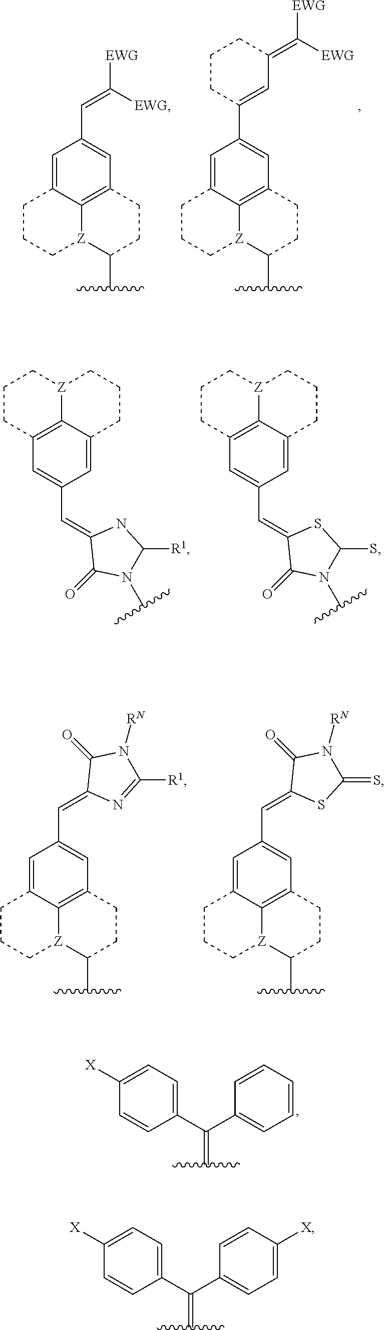 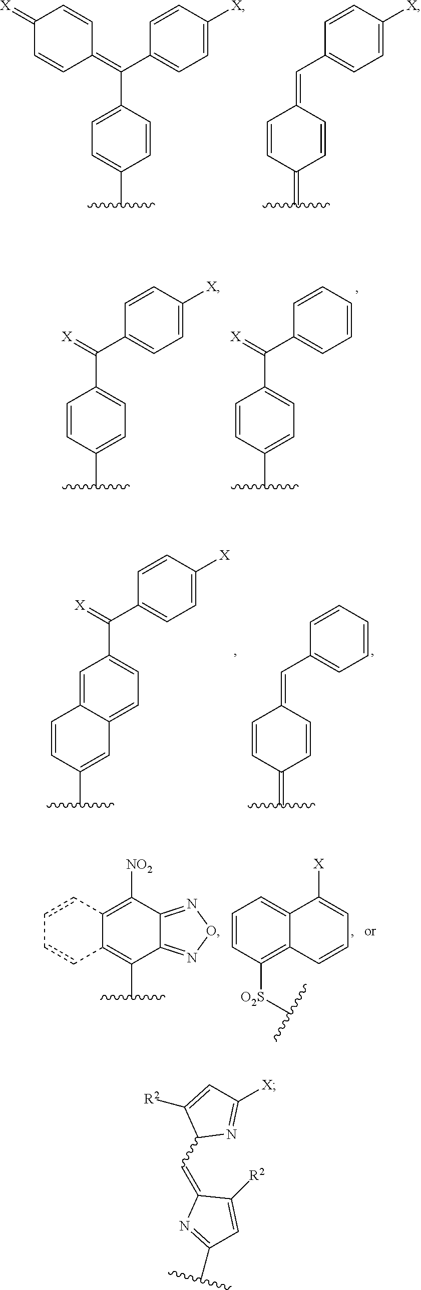
wherein:
each instance of EWG is independently an electron withdrawing group;
Z is N, NR^{N}, O, or S, as valency permits;
each instance of X is independently N(R^{N}), N(R^{N})₂, O, OR^{O}, S, or SR^{S}, as valency permits;
each instance of R¹ and R² is independently hydrogen, halogen, —CN, —NO₂, —N₃, —N(R^{N})₂, —OR^{O}, —SR^{S}, alkyl, alkenyl, alkynyl, carbocyclyl, aryl, heterocyclyl, heteroaryl, acyl, sulfinyl, or sulfonyl; and
each instance of R^{N}, R^{O}, and R^{S} is independently hydrogen, alkyl, alkenyl, alkynyl, carbocyclyl, aryl, heterocyclyl, heteroaryl, or acyl; and
wherein each formula is further optionally substituted.

30. The NTP of any one of the preceding claims, wherein the fluorogenic small molecule comprises one of the following:

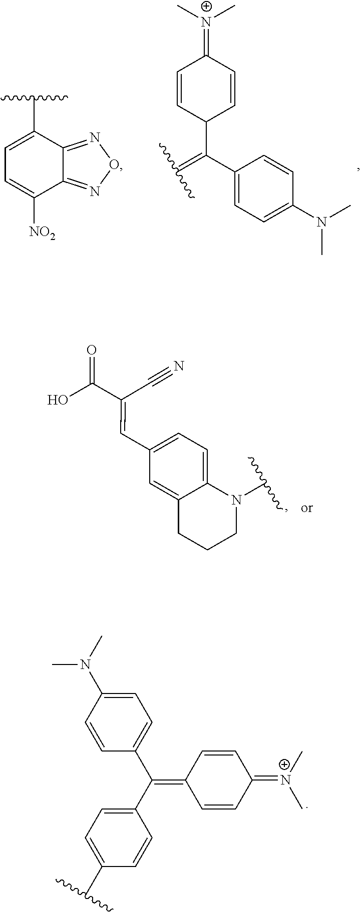

31. The NTP of any one of the preceding claims, wherein the NTP is selected from: 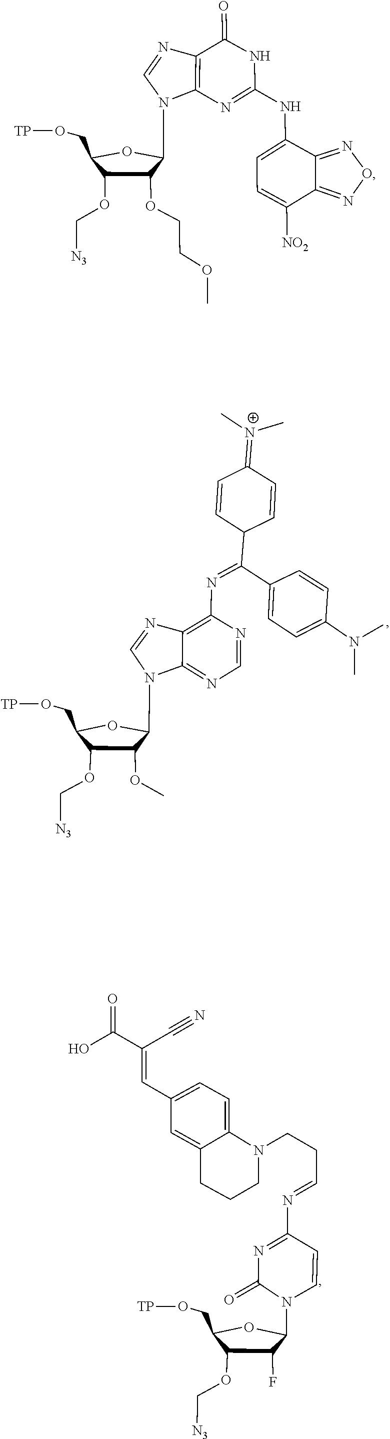 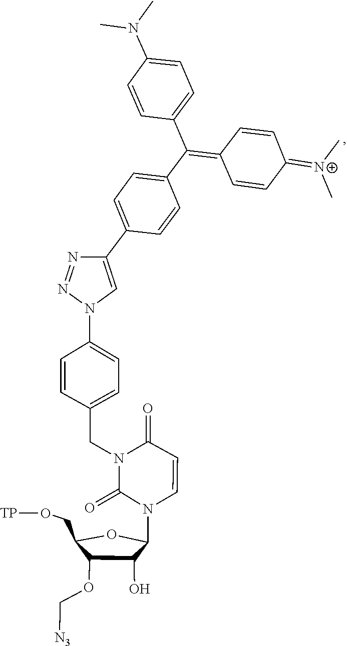
and salts thereof, wherein TP is a triphosphate group.

32. An RNA oligonucleotide comprising an NTP of any one of the preceding claims.

33. An RNA aptamer comprising an NTP of any one of the preceding claims.

34. A method for template-independent synthesis of an RNA oligonucleotide, the method comprising:
(a) providing an initiator oligonucleotide, wherein the initiator oligonucleotide is single-stranded RNA;
(b) providing a polymerase;
(c) combining the initiator oligonucleotide, the polymerase, and an NTP of any one of the preceding claims under conditions sufficient for the addition of the NTP to the 3′ end of the initiator oligonucleotide.

35. The method of claim 34, wherein the NTP comprises a 2′- and/or 3′-reversible terminator group and the method further comprises:
(d) deprotecting the 2′- and/or 3′-reversible terminator group at the 3′ end of the oligonucleotide formed in step (c).

36. The method of claim 35 further comprising:
(e) incorporating one or more nucleoside triphosphates to the 3′ end of the RNA oligonucleotide formed in step (d).

37. The method of any one of claims 34-36, wherein the RNA oligonucleotide is an RNA aptamer.

38. The method of any one of claims 34-37, wherein the polymerase is a poly(N) polymerase.

39. The method of claim 38, wherein the poly(N) polymerase is a poly(U) polymerase, poly(A) polymerase, poly(C) polymerase, or poly(G) polymerase; or a mutant thereof, or a homolog thereof.

40. The method of claim 38 or 39, wherein the poly(N) polymerase is a poly(U) polymerase, or a mutant thereof, or a homolog thereof.

41. The method of claim 40, wherein the poly(U) polymerase is wild-type Schizosaccharomyces pombe poly(U) polymerase, or a mutant thereof, or a homolog thereof.

42. The method of claim 41, wherein the poly(U) polymerase is wild-type Schizosaccharomyces pombe poly(U) polymerase.

43. The method of claim 41, wherein the poly(U) polymerase is a mutant of a wild-type Schizosaccharomyces pombe poly(U) polymerase, or a homolog thereof.

44. The method of any one of claims 34-43, wherein step (c) is carried out in the presence of a crowding agent.

45. The method of claim 44, wherein the crowding agent is polyethylene glycol (PEG).

46. The method of any one of claims 34-45, wherein step (c) is carried out in the presence of one or more additional enzymes.

47. The method of claim 46, wherein step (c) is carried out in the presence of an additional poly(N) polymerase.

48. The method of any one of claims 34-47, wherein step (c) is carried out in the presence of a yeast inorganic pyrophosphatase (PPI-ase).

49. The method of any one of claims 34-48, wherein step (c) is carried out in the presence of an RNase inhibitor.

50. The method of any one of claims 34-49, wherein step (c) is carried out in the presence of a non-hydrolyzable nucleoside.

51. The method of any one of claims 34-50, wherein the initiator oligonucleotide is covalently linked to a solid support.

52. The method of claim 51, wherein the initiator oligonucleotide is covalently linked to a solid support through a cleavable linker.

53. The method of any one of claims 34-52, wherein the initiator oligonucleotide is 5-20 nucleotides in length.

54. The method of any one of claims 34-53, wherein the initiator oligonucleotide is poly-rU, poly-rC, poly-rG, or poly-rA.

55. The method of any one of claims 34-53, wherein the initiator oligonucleotide comprises a 5′ cap.

56. The method of any one of claims 34-55 further comprising a step of isolating the resulting RNA oligonucleotide.

57. An RNA oligonucleotide prepared by the method according to any one of claims 34-56.

58. An RNA aptamer prepared by the method according to any one of claims 34-56.

59. A method of detecting a target comprising:
(i) contacting a target with an RNA aptamer of any one of the preceding claims; and
(ii) measuring or observing the fluorescence of the RNA aptamer and/or measuring or observing a change in the fluorescence lifetime of the RNA aptamer.

60. The method of claim 59, wherein a change in fluorescence and/or fluorescence lifetime is observed instantaneously after the contacting step.

61. The method of claim 60, wherein the change in fluorescence and/or fluorescence lifetime is observed within less than 1 second after the contacting step.

62. The method of claim 60, wherein change in fluorescence and/or fluorescence lifetime is observed within less than less than 2500, 2000, 1500, 1000, 750, 500, or 250 milliseconds (ms) after the contacting step.

63. The method of any one of claims 59-62, wherein an increase in fluorescence of at least 1-fold, 2-fold, 5-fold, 10-fold, 20-fold, 25-fold, 30-fold, 35-fold, 40-fold, 45-fold, 50-fold, 100-fold, 150-fold, 200-fold, 300-fold, 400-fold, or 500-fold is observed.

64. A kit comprising an NTP of any one of the preceding claims and/or an RNA aptamer of any one of the preceding claims.
